(19) United States
(12) Patent Application Publication
(10) Pub. No.: US 2026/0157858 A1
(43) Pub. Date: Jun. 11, 2026

(54) INTERVERTEBRAL DISC IMPLANTS HAVING VARYING ENDPLATE GEOMETRIES

(71) Applicant: CENTINEL SPINE, LLC, West Chester, PA (US)

(72) Inventors: Edward J. McShane, III, Collegeville, PA (US); Timothy J. Bertone, Philadelphia, PA (US); Damian Heinz, West Brandywine Township, PA (US); Thierry Marnay, Montpellier (FR)

(21) Appl. No.: 19/410,553

(22) Filed: Dec. 5, 2025

Related U.S. Application Data
(60) Provisional application No. 63/728,964, filed on Dec. 6, 2024.
(60) Provisional application No. 63/728,974, filed on Dec. 6, 2024.

(51) Int. Cl. A61F 2/44; A61F 2/30

(52) U.S. Cl. CPC A61F 2/4425; A61F 2/30771; A61F 2002/30153; A61F 2002/30245; A61F 2002/30275; A61F 2002/30322; A61F 2002/30843; A61F 2002/30884; A61F 2002/443

(57) ABSTRACT

Intervertebral disc implants having varying geometries for endplates, such as an upper endplate and a lower endplate which are dissimilar to one another, are provided. In one embodiment, a spinal implant comprises a first component having an outer surface for engaging one of the adjacent vertebrae, an inner surface opposite the outer surface and a fixation element extending from the outer surface. The implant further comprises a second component having an outer surface for engaging the other of the adjacent vertebrae, an inner surface opposite the outer surface and a fixation element extending from the outer surface. The fixation element of the first component has a different shape than the fixation element of the second component. This allows the implant to better conform to the anatomical space of certain patients, thereby increasing its ability to replicate the natural movement of the spine.

### CROSS-REFERENCE TO RELATED APPLICATIONS

This application claims the benefit of U.S. Provisional Application Ser. Nos. 63/728,982, 63/728,974 and 63/728,964, all of which were filed on Dec. 6, 2024, the complete disclosures of which are incorporated herein by reference for all purposes.

### TECHNICAL FIELD

Implantable spinal implants, more specifically intervertebral disc implants, and even more specifically artificial disc replacement implants having varying endplate geometries, such as top and bottom endplates which are dissimilar to one another, are described herein.

### BACKGROUND

Spinal instability is often attributed to undesirable excessive motion between vertebrae which can cause significant pain and morbidity. The instability may result from a number of causes, including abnormalities of the vertebrae, the intervertebral discs, the facet joints, or connective tissue around the spine. These abnormalities may arise from diseases, disorders or defects of the spine from trauma or bone degradation, such as osteoarthritis, or degenerative disc disease. When the spine becomes unstable, the vertebral column becomes misaligned and may allow micromotion between adjacent vertebrae. Vertebral misalignment and micromotion may result in wear to the vertebral bone surfaces and ultimately generate severe pain. These conditions are often chronic and create progressive problems for the sufferer.

Known treatments for spinal instability can include long-term medical management or surgery. Medical management is generally directed at controlling the symptoms, such as pain reduction, rather than correcting the underlying problem. For some patients, this may require chronic use of pain medications, which may alter the patient's mental state or cause other negative side effects. Surgical treatment typically includes decompression procedures to restore normal disc height, realign the column, and alleviate the pain.

Today, a variety of implantable spinal stabilization devices are available to address these spinal conditions or abnormalities in a more permanent manner than with decompression alone. Some of these implantable devices include intervertebral spinal stabilization devices that are configured for placement between adjacent vertebrae. These devices generally fall under the category of either fusion-promoting, or motion-preserving, and may be configured for specific segments of the spine such as the thoracic, lumbar or cervical region of the spine.

One such type of motion-preserving intervertebral spinal stabilization device is the artificial disc implant. Artificial disc implants may be configured as a total artificial disc implant intended to replace the entire disc between two adjacent vertebrae, or a partial artificial disc implant that replaces only a portion while leaving a native remnant of the disc intact. The procedure, known as disc arthroplasty, involves the insertion of an artificial intervertebral disc implant into the intervertebral space between adjacent vertebrae. Such a disc implant allows limited universal movement of the adjacent vertebrae with respect to each other. The aim of total disc replacement is to remove pain generation (caused by a degenerated disc), restore anatomy (disc height), and maintain mobility in the functional spinal unit so that the spine remains in an adapted sagittal balance. Sagittal balance is defined as the equilibrium of the trunk with the legs and pelvis to maintain harmonious sagittal curves and thus the damping effect of the spine. In contrast with fusion techniques, total disc replacement preserves mobility in the motion segment and mimics physiologic conditions.

As with many other types of implantable medical devices, it is believed that the more the artificial disc implant conforms to the patient, and specifically to the anatomical space it is intended to occupy, the better the implant will perform. What is desired are artificial disc replacement implants that can be easily manufactured for large scale production, reducing the wait time for patients to receive the implants and keeping the overall cost low, but also allow for customization to meet certain patient population needs for a better designed implant that can better conform to the patient's anatomy and consequently perform better clinically. Accordingly, artificial disc replacement implants that can address these unmet needs are desirable.

### SUMMARY

Intervertebral disc implants are provided that are configured for insertion in the disc space between two adjacent vertebral bodies. The implants may be configured for use anywhere along the spine as desired, including the cervical, lumbar or thoracic areas of the spine. These disc implants may be of the type that have an upper endplate and a lower endplate that cooperate with one another to provide an articulating joint between the endplates. The upper and lower endplates may be dissimilar, as will be described in more detail below, so that the disc implant has better anatomical conformity for certain patients. For instance, the upper and lower endplates may have different geometries with respect to each other, such as a different shape or size, or have different fixation elements. As will be described hereinbelow, various combinations of endplates with different geometries can be provided in a single spinal implant. The geometric configuration of the overall spinal implant can be selected and/or designed in accordance with factors to create a match to each patient's anatomy at each disc space, to ensure proper fixation and contact in relation to the condition of the patient's bony vertebral endplates (such as to account for damaged, degenerated, osteoporotic or diseased bone), to target the desired area of dense bone contact and fixation (e.g., selecting a geometric configuration where good bone purchase is possible, such as for example, the apophyseal ring, or by selecting a geometric configuration that would avoid damaging surrounding tissue), or selection of a geometric configuration to ensure a desired motion is achieved with the biomechanical properties of the spinal implant, thereby enabling better clinical outcomes.

In one aspect, an implant for insertion into an intervertebral space between two adjacent vertebrae comprises a first component having an outer surface for engaging one of the adjacent vertebrae and an inner surface opposite the outer surface. The first component comprises a fixation element extending from the outer surface. The implant further comprises a second component having an outer surface for engaging the other of the adjacent vertebrae and an inner surface opposite the outer surface. The second component comprises a fixation element extending from the outer surface. The fixation element of the first component has a different shape than the fixation element of the second component. This allows the surgeon to conform the implant to the anatomical space, thereby increasing its ability to duplicate the natural movement of the spine.

In various embodiments, the fixation element of the first and second components comprises one of a keel or a spike. In an exemplary embodiment, the fixation element of the first component comprises a spike and the fixation element of the second component comprises a keel.

In various embodiments, the first component is an upper or superior endplate and the second component is a lower or inferior endplate. In other embodiments, the second component is an upper endplate and the first component is a lower endplate.

In various embodiments, the fixation element of the first component comprises a spike and the fixation element of the second component comprises a first keel and a second keel. In one such embodiment, the first and second keels are laterally offset from a longitudinal axis of the implant. The first and second keels are preferably spaced laterally an equal distance from a longitudinal axis of the implant. In certain embodiments, the second component is an upper endplate and the first component is a lower endplate. In other embodiments, the first component is an upper endplate and the second component is a lower endplate.

In other embodiments, the fixation element of the first component comprises a spike and the fixation element of the second component comprises a central keel extending substantially along the longitudinal axis of the implant. The keel comprises first and second ends. The first end comprises a recess that opens upwardly and anteriorly. The recess may extend from the first end to a location between the first and second ends. In certain embodiments, the first end is a trailing end of the keels. The leading end of the keel may be rounded or beveled to facilitate insertion of the keels into cutouts formed in the adjacent vertebrae. In certain embodiments, the second component is an upper endplate and the first component is a lower endplate. In other embodiments, the first component is an upper endplate and the second component is a lower endplate.

In various embodiments, the fixation element of the first component comprises a spike and wherein the fixation element of the second component comprises a keel. The keel comprises a main body with first and second ends that extends along an axis of the endplate. The first end flares laterally outward from the main body of the keel and the second end may be rounded or beveled to facilitate insertion of the implant. In certain embodiments, the first end is a trailing end of the keel. In certain embodiments, the second component is an upper endplate and the first component is a lower endplate. In other embodiments, the first component is an upper endplate and the second component is a lower endplate.

In various embodiments, the implant has a longitudinal axis and the fixation element of the first component comprises a first set of least two spikes laterally offset from the longitudinal axis and a second set of at least two spikes laterally offset from the longitudinal axis on an opposite side of the longitudinal axis. In one such embodiment, the first set of spikes comprises at least two spikes offset from each other in a longitudinal direction. The two spikes may be laterally offset from each other. The offset may be equal or may be uneven relative to the longitudinal axis.

In one such embodiment, the spikes are arranged in first and second substantially symmetrical and substantially identically constructed groups. One group of spikes is disposed in a first lateral region of the endplate body, and the second group of spikes is disposed in a second lateral region of the endplate body opposite the longitudinal axis from the first group of spikes. Each lateral group may include a first or longitudinally forward or front spike, a second or longitudinally middle spike, and a third or longitudinally rear spike, such that the longitudinally middle spike is disposed longitudinally between the forward spike and the rear spike, and forward of the central lateral axis.

In another embodiment, at least some of the spikes within each lateral group are disposed on opposite sides of an axis extending laterally through a central or mid-line of implant. The central axis may be located substantially equal distances from the anterior and posterior ends of the prosthesis. In an exemplary embodiment, at least one spike is located on the posterior or leading region of the endplates and at least one spike is located in the anterior or trailing region of the endplates. At least one spike may be located on, or near, a central axis.

In another embodiment, each lateral group of spikes includes first and second longitudinally forward or front spikes, third and fourth longitudinally middle spikes, and fifth and sixth longitudinally rear spikes. The longitudinally middle spikes are disposed longitudinally between the forward spikes and the rear spikes, and forward of the central lateral axis. The longitudinally rear spikes are disposed rearward of the central lateral axis. The longitudinally middle spikes may be disposed on, or near, central axis.

In another aspect, an implant for insertion into an intervertebral space between two adjacent vertebrae is provided. The implant comprises a first component having an outer surface for engaging one of the adjacent vertebrae and an inner surface opposite the outer surface, the first component including a fixation element extending from the outer surface. The implant further comprises a second component having an outer surface for engaging the other of the adjacent vertebrae and an inner surface opposite the outer surface, the second component including a fixation element extending from the outer surface. The fixation element of the first component has a different shape than the fixation element of the second component. The fixation element of the first component comprise a keel and the fixation element of the second component comprises a keel.

In various embodiments, the fixation element of the first component comprises first and second keels laterally offset from a longitudinal axis of the implant. In one such embodiment, the fixation element of the second component comprises a keel having a main body with first and second ends that extends along an axis of the endplate. The first end flares laterally outward from the main body. In another embodiment, the fixation element of the second component comprises a keel, wherein the keel comprises first and second ends, wherein the first end comprises a recess extending from the first end to a location between the first and second ends. In one embodiment, the second component is an upper endplate and the first component is a lower endplate. In another embodiment, the first component is an upper endplate and the second component is a lower endplate.

In various embodiments, the fixation element of the first component comprises a keel and the keel comprises first and second ends. The first end comprises a recess extending from the first end to a location between the first and second ends. In one such embodiment, the fixation element of the second component comprises a keel having a main body with first and second ends that extends along an axis of the endplate. The first end flares laterally outward from the main body.

In various embodiments, the outer surface of the first component has a substantially domed shape. The outer surface may be generally convex from an anterior edge of each surface to a posterior edge of each surface. The outer surface may be generally convex from a first lateral edge of the surface to a second lateral edge opposite the first lateral edge. In this embodiment the entire upper surface of the first component is curved from its apex to the outer edges. The outer surface of the second component may have a substantially domed shape or it may be substantially flat. In an exemplary embodiment, the upper or superior component is domed and the lower or inferior component is substantially flat.

In another aspect, an implant for insertion into an intervertebral space between two adjacent vertebrae is provided. The implant comprises a first component having an outer surface for engaging one of the adjacent vertebrae and an inner surface opposite the outer surface, the first component including a fixation element extending from the outer surface. The implant further comprises a second component having an outer surface for engaging the other of the adjacent vertebrae and an inner surface opposite the outer surface, the second component including a fixation element extending from the outer surface. The fixation element of the first component has a different shape than the fixation element of the second component.

In various embodiments, the fixation element of the first component comprises at least one spike and at least one keel. In some embodiments, the fixation element of the second component comprises at least one spike and at least one keel. In one such embodiment, the implant includes a first keel extending from the upper surface of upper endplate and second and third keels extending from the lower bone contacting surface of lower endplate. The first keel may extend centrally along the longitudinal axis of the implant and the second and third keels may generally extend parallel to the longitudinal axis, but are laterally offset from this axis. The spikes are disposed laterally outward from the central keel on the first component. In an exemplary embodiment, the implant includes a group of spikes on each side of the central keel.

In various embodiments, the fixation element of the first and second components each comprise a keel and the keel of the first component is laterally offset from the keel of the second component. The fixation element of the first component is laterally offset from the longitudinal axis of the implant and the fixation element of the second component is laterally offset from the longitudinal axis on an opposite side of the longitudinal axis from the fixation element of the first component.

In various embodiments, the first and/or the second component comprises first and second keels laterally offset from the longitudinal axis of the endplate. The offset may be even or uneven relative to the longitudinal axis. The keels are oriented at a transverse angle relative to the bone contacting surface. In one such embodiment, the keels extend in a laterally outward direction such that the outer surface of the keels is disposed laterally outward from the base of the keels. In another embodiment, the keels extend in a laterally inward direction such that the outer surface of the keels is laterally inward from the base of the keels.

In various embodiment, the fixation element on the first component is larger than the fixation element on the second component. In one such embodiment, the fixation elements comprise keels. The keel of the second component may have a larger width, length and/or depth than the keel of the first component. In another embodiment, fixation element of the first component comprises first and second keels with first and second widths and the fixation element of the second component comprises a keel with a third width. The third width is greater than the first and second widths. The first width may be equal to, or greater than, the second width.

In various embodiments, the first component comprises first, second and third keels. The first keel extends along a longitudinal axis of the first component and the second and third keels are laterally offset from the longitudinal axis. In one such embodiment, the first keel extends a first distance away from the first component and the second and third keels extend a second and third distance, respectively, away from the first component. The first distance is greater than the second and third distances. In addition, or alternatively, the first, second and third keels have first, second and third widths, respectively, and the first width is greater than the second and third widths. In addition, or alternatively, the first, second and third keels have first, second and third lengths, respectively, and the first length is greater than the second and third lengths.

In various embodiments, the first component has first and second keels non-uniformly spaced across the outer surface of the first component. In one such embodiment, the second component has first and second keels non-uniformly spaced across the outer surface of the second component.

In various embodiments, an outer surface of the first component has a first area, and the outer surface of the second component has a second area. In one such design, the first area can be less than the second area. In another design, the first area can be greater than the second area. In one embodiment, the first component is an upper endplate and the second component is a lower endplate. The fixation element of the first component may be one or more keels and/or one or more spikes. The fixation element of the second component may be one or more keels and/or one or more spikes.

In another aspect, an implant for insertion into an intervertebral space between two adjacent vertebrae is provided. The implant comprises a first component having an outer surface for engaging one of the adjacent vertebrae and an inner surface opposite the outer surface. The first component includes a fixation element extending from the outer surface. The implant further comprises a second component having an outer surface for engaging the other of the adjacent vertebrae and an inner surface opposite the outer surface. The second component includes a fixation element extending from the outer surface. The fixation element of the first component can have a different shape than the fixation element of the second component. The outer surface of one of the first and second components can have a substantially domed shape. These domed shapes may differ between the two endplates. For example, in one embodiment, the implant can include a lesser domed inferior plate mated with a larger domed superior plate.

In various embodiments, the outer surface of the other of the first and second components is substantially flat. In this embodiment, the domed endplate may be the upper endplate or the lower endplate. Alternatively, the outer surface of both the first and second components has a substantially domed shape.

In various embodiments, the outer surface of the upper endplate extends from an anterior edge to a posterior edge opposite the anterior edge and is substantially convex from the anterior edge to the posterior edge. In other embodiments, outer surface of the upper endplate extends from a first lateral edge to a second lateral edge opposite the first lateral edge and is substantially convex from the first lateral edge to the second lateral edge. Thus, the entire outer surface of the upper endplate is curved from the apex to the outer edges of the endplate.

In various embodiments, the fixation element of the first component comprises a spike and the fixation element of the second component comprises a keel. In certain embodiments, the fixation element of the first component comprises a spike and the fixation element of the second component comprises a first keel and a second keel. In other embodiments, the fixation element of the first component comprises a keel and the fixation element of the second component comprises a first keel and a second keel.

In various embodiments, the fixation element of the first component comprises first and second keels laterally offset from a longitudinal axis of the implant. In one such embodiment, the fixation element of the second component comprises a keel having a main body with first and second ends that extends along an axis of the endplate. The first end flares laterally outward from the main body. In other embodiments, the fixation element of the second component comprises a keel having first and second ends. The first end comprises a recess extending from the first end to a location between the first and second ends. In yet another embodiment, the fixation element of the second component comprises a keel having a main body with first and second ends that extends along an axis of the endplate. The first end flares laterally outward from the main body.

In another aspect, an implant for insertion into an intervertebral disc space between two adjacent vertebrae is provided. The implant comprises a first endplate having an outer surface for engaging one of the adjacent vertebrae and an inner surface opposite the outer surface, the first endplate including a first fixation element extending from the outer surface and a second endplate having an outer surface for engaging the other of the adjacent vertebrae and an inner surface opposite the outer surface, the second endplate including a second fixation element extending from the outer surface. The first endplate has a first height in a direction substantially perpendicular to the longitudinal axis and the first fixation element has a second height in a direction substantially perpendicular to the longitudinal axis. The ratio between the first height and the second height is about 4:3 to about 2:1.

In various embodiments, the second endplate has a third height in a direction substantially perpendicular to the longitudinal axis and the second fixation element has a fourth height in a direction substantially perpendicular to the longitudinal axis. The ratio between the third height and the fourth height is about 2:1 to about 1:1.

In various embodiments, the first fixation element comprises a keel. The second fixation element may also comprise a keel. Applicant has discovered that a shallower keel can provide sufficient purchase and fixation within the vertebral bone without requiring the removal of an excessive amount of bone. In particular, it has been found that a deeper keel does not necessarily improve implant stability or resistance to migration, and in some cases may undesirably weaken the surrounding bone structure or compromise endplate integrity.

In another aspect, an implant for insertion into an intervertebral disc space between two adjacent vertebrae is provided. The implant comprises a first endplate having an outer surface for engaging one of the adjacent vertebrae and an inner surface opposite the outer surface, the first endplate including a first fixation element extending from the outer surface and a second endplate having an outer surface for engaging the other of the adjacent vertebrae and an inner surface opposite the outer surface, the second endplate including a second fixation element extending from the outer surface. The first endplate has a first length in a direction substantially parallel to the longitudinal axis and the first fixation element has a second length in a direction substantially parallel to the longitudinal axis, wherein a ratio between the first length and the second length is about 4:3 to about 2:1.

In various embodiments, the second endplate has a third length in a direction substantially parallel to the longitudinal axis and the second fixation element has a fourth length in a direction substantially parallel to the longitudinal axis. The ratio between the third length and the fourth length is about 4:3 to about 2:1.

In various embodiments, the first fixation element comprises a keel. The second fixation element may also comprise a keel.

In another aspect, an implant for insertion into an intervertebral disc space between two adjacent vertebrae is provided. The implant comprises a first endplate having an outer surface for engaging one of the adjacent vertebrae and an inner surface opposite the outer surface, the first endplate including a first fixation element extending from the outer surface and a second endplate having an outer surface for engaging the other of the adjacent vertebrae and an inner surface opposite the outer surface, the second endplate including a second fixation element extending from the outer surface. The first endplate has a first width in a direction substantially perpendicular to the longitudinal axis and the first fixation element has a second width in a direction substantially perpendicular to the longitudinal axis, wherein a ratio between the first width and the second width is about 3:8.

In various embodiments, the second endplate has a third width in a direction substantially perpendicular to the longitudinal axis and the second fixation element has a fourth width in a direction substantially perpendicular to the longitudinal axis, wherein a ratio between the third width and the fourth width is about 3:8.

In various embodiments, the first fixation element comprises a keel. The second fixation element may also comprise a keel.

### BRIEF DESCRIPTION OF THE DRAWINGS

The accompanying drawings, which are incorporated in and constitute a part of this specification, illustrate several embodiments of the disclosure and together with the description, serve to explain the principles of the disclosure.

FIGS. 1A-1E are perspective views of representative spinal implants.

FIG. 2A is a perspective view of one embodiment of a spinal implant with keels having different shapes on the upper and lower endplates;

FIG. 2B illustrates another embodiment of a spinal implant with two keels on a lower endplate and a single keel on an upper endplate;

FIG. 3A illustrates another embodiment of a spinal implant with keels having different shapes on upper and lower endplates;

FIG. 3B illustrates another embodiment of a spinal implant with two keels on an upper endplate and a single keel on the lower endplate;

FIG. 4A illustrates another embodiment of a spinal implant with keels having different shapes on upper and lower endplates;

FIG. 4B illustrates another embodiment of a spinal implant with keels having different shapes on upper and lower endplates;

FIG. 5A illustrates another embodiment of a spinal implant with spikes on a lower endplate and a keel on the upper endplate;

FIG. 5B illustrates another embodiment of a spinal implant with spikes on an upper endplate and a keel on the lower endplate;

FIG. 6A illustrates another embodiment of a spinal implant with keels having different shapes on upper and lower endplates;

FIG. 6B illustrates another embodiment of a spinal implant with two keels on an upper endplate and a single keel on the lower endplate;

FIG. 7A illustrates another embodiment of a spinal implant with keels having different shapes on upper and lower endplates;

FIG. 7B illustrates another embodiment of a spinal implant with two keels on a lower endplate and a single keel on the upper endplate;

FIG. 8A illustrates another embodiment of a spinal implant with spikes on the lower endplate and a keel on the upper endplate;

FIG. 8B illustrates another embodiment of a spinal implant with spikes on the lower endplate and two keels on the upper endplate;

FIG. 9A illustrates another embodiment of a spinal implant with spikes on the upper endplate and a keel on the lower endplate;

FIG. 9B illustrates another embodiment of a spinal implant with spikes on the upper endplate and two keels on the lower endplate;

FIGS. 10A-10D illustrate an endplate for a spinal implant having three keels;

FIG. 11A illustrates another embodiment of a spinal implant with three keels on the lower endplate and a single keel on the upper endplate;

FIG. 11B illustrates another embodiment of a spinal implant with three keels on the lower endplate and a single keel on the upper endplate;

FIGS. 12A-12D illustrate another embodiment of an endplate for a spinal implant having three keels;

FIGS. 13A-13C illustrate various embodiments of spinal implants having keels with different widths or thicknesses;

FIGS. 14A and 14B illustrate another embodiment of a spinal implant with spikes on the upper and lower endplates;

FIGS. 15A and 15B illustrate another embodiment of a spinal implant with spikes on the upper and lower endplates;

FIG. 16 illustrates another embodiment of a spinal implant with the offset keels on the upper and lower endplates;

FIGS. 17A and 17B illustrate various embodiments of a spinal implant with medially and laterally angled keels;

FIG. 18 illustrates an embodiment of a spinal implant with medially and laterally angled spikes;

FIGS. 19A-19D illustrates another embodiment of a spinal implant with a domed upper endplate;

FIGS. 20A-20D illustrates another embodiment of a spinal implant with a domed upper endplate;

FIG. 21A illustrates an embodiment of a spinal implant with a larger lower endplate and a keel on the upper and lower endplates;

FIG. 21B illustrates an embodiment of a spinal implant with a larger lower endplate and spikes on the upper and lower endplates;

FIG. 22 illustrates an embodiment of a spinal implant with both spikes and keels on the upper and lower endplates;

FIG. 23A illustrates an embodiment of a spinal implant with a keel and spikes on the upper endplate and a keel having a different shape on the lower endplate;

FIG. 23B illustrates an embodiment of a spinal implant with a keel and spikes on the upper endplate and a keel having a different shape on the lower endplate;

FIG. 23C illustrates an embodiment of a spinal implant with a keel and spikes on the upper endplate and two keels having a different shape on the lower endplate;

FIG. 23D illustrates an embodiment of a spinal implant with a keel and spikes on the upper endplate and a keel having a different shape on the lower endplate;

FIG. 23E illustrates an embodiment of a spinal implant with a keel and spikes on the upper endplate and a keel having a plurality of spikes on the lower endplate;

FIG; 24A illustrates the upper endplate of FIG. 1E with a keel having a smaller height;

FIG. 24B illustrates the upper endplate of FIG. 1A with a keel having a smaller height;

FIG. 25A illustrates the upper endplate of FIG. 1E with a keel having a smaller width;

FIG. 25B illustrates the upper endplate of FIG. 1A with a keel having a smaller width;

FIG. 26A illustrates the upper endplate of FIG. 1E with a keel having a shorter length;

FIG. 26B illustrates the upper endplate of FIG. 1A with a keel having a shorter length; and

FIGS. 27A-27D illustrate alternative embodiments with various fixation spike positions of the upper endplate of FIG. 1E.

### DESCRIPTION OF THE EMBODIMENTS

This description and the accompanying drawings illustrate exemplary embodiments and should not be taken as limiting, with the claims defining the scope of the present disclosure, including equivalents. Various mechanical, compositional, structural, and operational changes may be made without departing from the scope of this description and the claims, including equivalents. In some instances, well-known structures and techniques have not been shown or described in detail so as not to obscure the disclosure. Like numbers in two or more figures represent the same or similar elements. Furthermore, elements and their associated aspects that are described in detail with reference to one embodiment may, whenever practical, be included in other embodiments in which they are not specifically shown or described. For example, if an element is described in detail with reference to one embodiment and is not described with reference to a second embodiment, the element may nevertheless be claimed as included in the second embodiment. Moreover, the depictions herein are for illustrative purposes only and do not necessarily reflect the actual shape, size, or dimensions of the system or illustrated components.

It is noted that, as used in this specification and the appended claims, the singular forms “a”, “an”, and “the”, and any singular use of any word, include plural referents unless expressly and unequivocally limited to one referent. As used herein, the term “include” and its grammatical variants are intended to be non-limiting, such that recitation of items in a list is not to the exclusion of other like items that can be substituted or added to the listed items.

Spinal devices are provided that are configured for implantation within an intervertebral space disposed between vertebral bodies. The vertebral bodies can be anatomically adjacent vertebral bodies, or can be the vertebral bodies that remain after a discectomy has been performed that removed a vertebral body from a location between the vertebral bodies. The devices are configured to be inserted into the intervertebral space, and achieve restoration of height while maintaining mobility. The intervertebral space can be disposed anywhere along the spine as desired, including the cervical, lumbar or thoracic areas of the spine. The implants described herein are particularly useful for conforming a specific implant to a particular anatomical space, thereby increasing the ability of the implant to duplicate the natural movement of the spine.

Certain terminology is used in the following description for convenience only and is not limiting. The words “right”, “left”, “lower” and “upper” designate directions in the drawings to which reference is made. The words “inner” or “distal” and “outer” or “proximal” refer to directions toward and away from, respectively, the geometric center of the implant and related parts thereof. The words “anterior”, “posterior”, “superior”, “inferior”, “medial”, “lateral”, and related words and/or phrases designate preferred positions and orientations in the human body to which reference is made and are not meant to be limiting. The terminology includes the above listed words, derivatives thereof and words of similar import.

Unless otherwise specified herein, the terms “lateral”, “longitudinal”, and “transverse” are used to describe the orthogonal directional components of various components. It should be appreciated that while the longitudinal and lateral directions are illustrated as extending along a horizontal plane, and that the transverse direction is illustrated as extending along a vertical plane, the planes that encompass the various directions may differ during use. For instance, when the implant is implanted into an intervertebral space, such as the intervertebral space, the transverse direction extends generally along the superior-inferior (or caudal-cranial) direction, while the plane defined by the longitudinal direction and lateral directions generally in the anatomical plane defined by the anterior-posterior direction, and the medial-lateral direction. Accordingly, the directional terms “vertical” and “horizontal” are used to describe the implants and their components as illustrated merely for the purposes of clarity and illustration.

As mentioned, intervertebral disc implants are provided that are configured for insertion in the disc space between two adjacent vertebral bodies. The implants may be configured for use anywhere along the spine as desired, including the cervical, lumbar or thoracic areas of the spine. These disc implants may be of the type that have an upper endplate and a lower endplate that cooperate with one another to provide an articulating joint between the endplates. The upper and lower endplates may be dissimilar, as will be described in more detail below, so that the disc implant has better anatomical conformity for certain patients. For instance, the upper and lower endplates may have different geometries with respect to each other, such as a different shape or size, or have different fixation elements. As will be described hereinbelow, various combinations of endplates with different geometries can be provided in a single spinal implant. The geometric configuration of the overall spinal implant can be selected and/or designed in accordance with factors to create a match to each patient's anatomy at each disc space, to ensure proper fixation and contact in relation to the condition of the patient's bony vertebral endplates (such as to account for damaged, degenerated, osteoporotic or diseased bone), to target the desired area of dense bone contact and fixation (e.g., selecting a geometric configuration where good bone purchase is possible, such as for example, the apophyseal ring, or by selecting a geometric configuration that would avoid damaging surrounding tissue), or selection of a geometric configuration to ensure a desired motion is achieved with the biomechanical properties of the spinal implant, thereby enabling better clinical outcomes.

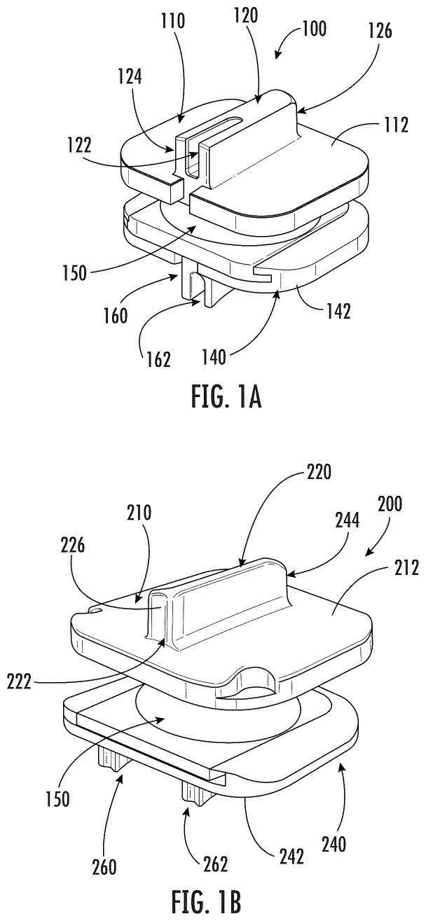

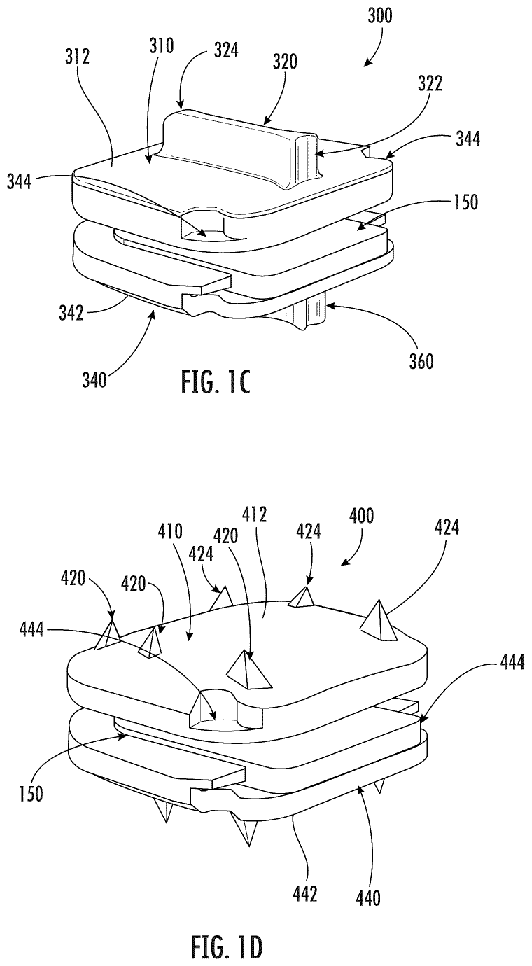

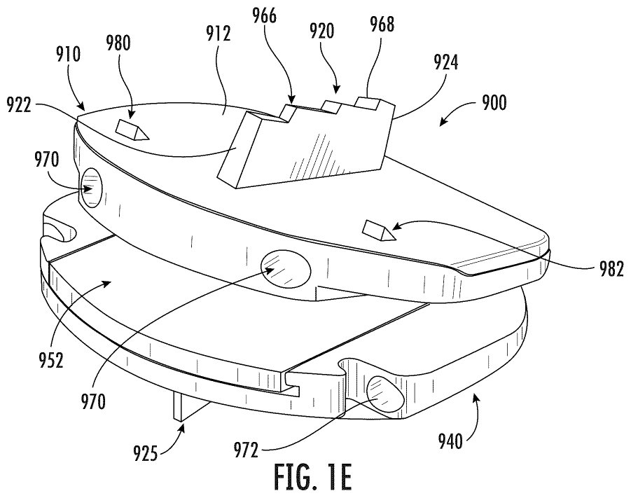

Referring now to FIGS. 1A-1E, certain representative prior art devices will be described for general reference to the inventive concepts discussed below. These devices are configured for implantation within an intervertebral space disposed between vertebral bodies and may be particularly useful for implantation between adjacent cervical vertebral bodies. As shown in FIG. 1A, a spinal implant 100 comprises an upper or superior endplate 110, a lower or inferior endplate 140 and a core 150. Superior endplate 110 rides upon the core 150 and is operable to rotate relative to core 150, including torsional rotation about a vertical axis, flexion/extension rotation in the posterior/anterior directions and lateral bending rotation. Core 150 includes a substantially spherical dome having a convex upper surface that contacts an internal concave surface of upper endplate 110 and an inlay that is slidably disposed within longitudinal recesses or slots in lower endplate 140. For example, the inlay may include a plastic snap-in projection that fits within snap-in recesses or slots in lower endplate 140 such that the plastic inlay can snap into place but is thereafter inhibited from being removed. The spherical dome may be a separate element from the inlay and coupled thereto, or the core and inlay may be manufactured as a single, integral element.

In some embodiments, there are no marked angles that separate the dome from the endplate surface. Instead, the outer surface of the endplate may be continuous and smooth. For example, the outer surface of the endplate can be seen as having a convex radius from edge to edge swept from front to back, and a separate, and possibly different radius, convex surface swept edge to edge from right to left.

The upper bone contacting surface 112 of upper endplate 110 is substantially flat and the side surfaces of upper endplate 110 are substantially perpendicular to the upper surface and include rounded or beveled corners. Similarly, the lower bone contacting 142 surface of lower endplate 140 is substantially flat and the side surfaces of lower endplate 140 are substantially perpendicular to the lower surface and include rounded or beveled corners.

Implant 100 further includes a first keel 120 extending from upper bone contacting surface 112 of upper endplate 110 and a second keel 160 extending from lower bone contacting surface 142 of lower endplate 140. Keels 120, 160 are centrally located on endplates 110, 140 such that they generally extend along a longitudinal axis of the endplates. The longitudinal axis is hereafter defined as the axis generally extending from an anterior portion of the implant to a posterior portion of the prosthesis after insertion of the implant into the intervertebral body. The anterior portion may also be considered the trailing end of the prosthesis and the posterior portion the leading end (with reference to the direction the prosthesis is introduced into the intervertebral space).

Keel 120 includes a recess 122 that opens upwardly and anteriorly and keel 160 includes a recess 162 that opens downwardly and anteriorly. Recesses 122, 162 extend from a trailing end 124 of keels 120, 160 to a portion of keels 120, 160 located between the trailing and leading ends 124, 126 of keels 120, 160, preferably about 25% to about 75% of the distance between the leading and trailing ends, or about 50%. The recesses 122, 162 extend from the bone contacting surfaces 112, 142 of endplates 110, 140 to the upper surface of keels 120, 160 and generally have a width of about 20% to about 50% of the overall width of the keels.

The trailing end 124 and the side surfaces of keels 120, 160 are substantially perpendicular to the bone contacting surfaces of endplates 110, 140. The upper surface of keels 120, 160 are substantially parallel to the bone contacting surfaces of endplates 110, 140. The leading ends 126 of the keels 120, 160 are rounded or beveled to facilitate insertion of the keels into cutouts formed in the adjacent vertebrae.

Upper and lower endplates 110, 140 and components thereof, can be formed of any suitable biocompatible material, such as cobalt chromium molybdenum (CoCrMo), titanium and titanium alloys, stainless steel, ceramics, or polymers such as polyetheretherketone (PEEK), polyetherketoneketone (PEKK), bioresorbable materials, and bone graft (for example, allografts and xenografts). A coating may be added or applied to the endplates to improve physical or chemical properties. The coatings may help to ensure bony in or on growth or medication. Examples of coatings include plasma-sprayed titanium coating or hydroxyapatite. The porosity of the coating ideally permits vascularization and osteoblast formation with subsequent bony on-growth.

Implant 100 is manufactured and sold by Centinel Spine under the tradename of Prodisc® C. A more complete description of implant 100 can be found in commonly assigned, U.S. Pat. No. 7,204,852, the complete disclosure of which is incorporated herein by reference for all purposes.

Referring now to FIG. 1B, a spinal implant 200 comprises an upper or superior endplate 210, a lower or inferior endplate 240 and a core 150. Superior endplate 210 rides upon the core 150 and is operable to rotate relative to core 150, including torsional rotation about a vertical axis, flexion/extension rotation in the posterior/anterior directions and lateral bending rotation. Similar to implant 100, core 150 includes a substantially spherical dome having a convex upper surface that contacts an internal concave surface of upper endplate 210 and an inlay that is slidably disposed within longitudinal recesses or slots in lower endplate 240.

The upper bone contacting surface 212 of upper endplate 210 is substantially flat and the side surfaces of upper endplate 210 are substantially perpendicular to the upper surface and include rounded or beveled corners. Similarly, the lower bone contacting surface 242 of lower endplate 240 is substantially flat and the side surfaces of lower endplate 240 are substantially perpendicular to the lower surface and include rounded or beveled corners.

Implant 200 further includes a first keel 220 extending from the upper surface 212 of upper endplate 210 and second and third keels 260, 262 extending from the lower bone contacting surface 242 of lower endplate 240. First keel 220 generally extends centrally along the longitudinal axis of implant 200 and second and third keels 260, 262 generally extend parallel to the longitudinal axis, but are laterally offset from this axis. Keels 260, 262 are preferably spaced laterally an equal distance from a longitudinal axis of the implant 200.

Keels 220, 260, 262 each include an anterior or trailing end 222 that flares laterally outward to anchor this end of the keels in their cutouts in adjacent vertebrae. Trailing end 222 may form a concave surface 226 facing in the trailing direction. The posterior or leading end 224 of the keels 220, 260, 262 are rounded or beveled to facilitate insertion of the keels into cutouts formed in the adjacent vertebrae. Keels 220, 260, 262 each extend along the bone contacting surfaces such that a space exists between the anterior and posterior surfaces of the bone contacting surfaces of endplates 210, 240 and the keels. The side surfaces of keels 220, 260, 262 are substantially perpendicular to the bone contacting surfaces of endplates 210, 240, but, in some embodiments, may also taper inward in the directions away from these bone contacting surfaces such that the upper surface of keels 220, 260, 262 has a smaller width than the base. The upper surfaces of keels 220, 260, 262 are preferably rounded or beveled to create a smooth curved transition from the side surfaces to the top surfaces.

Implant 200 is manufactured and sold by Centinel Spine of West Chester, Pennsylvania under the tradename of Prodisc® C Nova. A more complete description of implant 200 can be found in commonly assigned U.S. Pat. No. 8,998,990, the complete disclosure of which is incorporated herein by reference for all purposes.

Referring now to FIG. 1C, a spinal implant 300 comprises an upper or superior endplate 310, a lower or inferior endplate 340 and a core 150. Superior endplate 310 rides upon the core 350 and is operable to rotate relative to core 350, including torsional rotation about a vertical axis, flexion/extension rotation in the posterior/anterior directions and lateral bending rotation. Similar to previously described devices, core 150 includes a substantially spherical dome having a convex upper surface that contacts an internal concave surface of upper endplate 310 and an inlay that is slidably disposed within longitudinal recesses or slots in lower endplate 340.

The upper contact surface 312 of upper endplate 310 is substantially flat and the side surfaces of upper endplate 310 are substantially perpendicular to the upper surface 312 and include rounded or beveled corners. Similarly, the lower contact surface 342 of lower endplate 340 is substantially flat and the side surfaces of lower endplate 340 are substantially perpendicular to lower surface 342 and include rounded or beveled corners. Upper and lower endplates 310, 340 have recesses or notches 344 carved into the two anterior corners.

Implant 300 further includes a first central keel 320 extending from the upper bone contacting surface 312 of upper endplate 310 and a second central keel 360 extending from the lower bone contacting surface 342 of lower endplate 240. Keels 320, 360 each include an anterior or trailing end 322 that flares laterally outward to anchor this end of the keels in their cutouts in adjacent vertebrae. Keels 320, 360 each extend along the bone contacting surfaces such that a space exists between the anterior and posterior surfaces of the bone contacting surfaces of endplates 310, 340 and the keels. The sides surfaces of keels 320, 360 are substantially perpendicular to the bone contacting surfaces of endplates 310, 340, but, in some embodiments, may also taper inward in the directions away from these bone contacting surfaces such that the upper portion of keels 320, 360 has a smaller width than the base. The top surfaces of keels 320, 360 are preferably rounded or beveled to create a smooth curved transition from the side surfaces to the top surfaces. The posterior or leading end 324 of the keels 320, 360 are beveled to facilitate insertion of the keels into cutouts formed in the adjacent vertebrae.

Implant 300 is manufactured and sold by Centinel Spine of West Chester, Pennsylvania under the tradename of Prodisc® C SK. A more complete description of implant 300 can be found in commonly assigned U.S. Pat. No. 8,998,990, the complete disclosure of which is incorporated herein by reference for all purposes.

Referring now to FIG. 1D, a spinal implant 400 comprises an upper or superior endplate 410, a lower or inferior endplate 440 and a core 150. Superior endplate 410 rides upon the core 150 and is operable to rotate relative to core 150, including torsional rotation about a vertical axis, flexion/extension rotation in the posterior/anterior directions and lateral bending rotation. Core 150 includes a substantially spherical dome having a convex upper surface that contacts an internal concave surface of upper endplate 410 and an inlay that slidably disposed within longitudinal recesses or slots in lower endplate 440.

The upper bone contacting surface 412 of upper endplate 410 and the lower bone contacting surface 442 of lower endplate 440 have a substantially domed shape such that the entire contact surfaces are curved. Thus, the contact surfaces are generally convex from an anterior edge of each surface to the posterior edge of each surface. Similarly, the contact surfaces are generally convex from a first lateral edge of the surface to a second lateral edge opposite the first lateral edge. The overall contact surfaces are curved from an apex of the surface to its exterior edges. The side surfaces of upper and lower endplates 410, 440 are disposed substantially perpendicularly to the upper surfaces and include rounded or beveled corners. Upper and lower endplates 410, 440 have recesses or notches 444 carved into the two anterior corners.

Upper and lower endplates 410, 440 each include at least one spike, such as a plurality of spikes, projecting from the bone facing surfaces 412, 442 of the endplate bodies. The spikes are arranged in first and second substantially symmetrical and substantially identically constructed groups. One group of spikes 424 is disposed in a first lateral region of the endplate body, and the second group of spikes 420 is disposed in a second lateral region of the endplate body opposite the longitudinal axis from the first group of spikes 424. Each lateral group 420, 424 includes a first or longitudinally forward or front spike, a second or longitudinally middle spike, and a third or longitudinally rear spike, such that the longitudinally middle spike is disposed longitudinally between the forward spike and the rear spike, and forward of the central lateral axis. The spikes of each lateral group can be constructed substantially identically and symmetrically. In some embodiments, the spikes in each group are laterally spaced from each other relative to the longitudinal axis.

Each spike has a substantially pyramidal shape and extends up from a base having a triangular or alternatively shaped footprint at the bone facing surface, to an upper or outer transverse tip. Each surface extends between the base and the tip, and can be connected between the base and the tip as illustrated. The spikes thus define a transverse axis that extends transversely between the outer tip and the bone facing surface. The spikes define recesses therein that extend from a portion of spikes between the base and the tip and into, but not through, endplates 410, 440.

Implant 400 is manufactured and sold by Centinel Spine of West Chester, Pennsylvania under the tradename of Prodisc® C Vivo. A more complete description of implant 400 can be found in commonly assigned U.S. Pat. No. 8,858,636, the complete disclosure of which is incorporated herein by reference for all purposes.

Referring now to FIG. 1E, a spinal implant 900 comprises an upper or superior endplate 910, a lower or inferior endplate 940 and a core 952. Superior endplate 910 rides upon the core 952 and is operable to rotate relative to core 952, including torsional rotation about a vertical axis, flexion/extension rotation in the posterior/anterior directions and lateral bending rotation. Core 952 includes a substantially spherical dome (not shown) having a convex upper surface that contacts an internal concave surface (not shown) of superior endplate 910 and an inlay that is slidably disposed within a longitudinal recesses or slot in inferior endplate 910. The inlay may include a plastic snap-in projection that fits within snap-in recesses or slots of the recess of inferior endplate 940 such that the plastic inlay can snap into place but is thereafter inhibited from being removed. The spherical dome may be a separate element from the inlay and coupled thereto, or the core and inlay may be manufactured as a single, integral element.

Upper and inferior endplates 910, 940 and endplates thereof, can be formed of any suitable biocompatible material, such as cobalt chromium molybdenum (CoCrMo), titanium and titanium alloys, stainless steel, ceramics, or polymers such as polyetheretherketone (PEEK), polyetherketoneketone (PEKK), bioresorbable materials, and bone graft (for example, allografts and xenografts). A coating may be added or applied to the endplates to improve physical or chemical properties. The coatings may help to ensure bony in or on growth or medication. Examples of coatings include plasma-sprayed titanium coating or hydroxyapatite. The porosity of the coating ideally permits vascularization and osteoblast formation with subsequent bony on-growth.

Core 952, and particularly the spherical dome, is generally formed of a biocompatible, wear-resistant material suitable for articulation against the metallic surface of the superior endplate 910. In some embodiments, the core 952 may comprise an ultra-high molecular weight polyethylene (UHMWPE) or other medical-grade polymer having a low coefficient of friction to facilitate smooth motion between the articulating surfaces. In other embodiments, the core may be formed of a ceramic material, such as alumina or zirconia, or a composite material combining polymeric and ceramic constituents to optimize wear characteristics and load distribution. The selection of material for core 952 may depend on the desired articulation performance, mechanical strength, and compatibility with the materials of the endplates.

In some embodiments, upper bone contacting surface 912 of superior endplate 910 is substantially planar and the side surfaces of superior endplate 910 are substantially perpendicular to the upper surfaces. The side surfaces may include straight rounded or beveled corners. Similarly, the lower bone contacting surface of inferior endplate 940 may be substantially planar and the side surfaces of inferior endplate 940 are substantially perpendicular to the lower surface and may include straight rounded or beveled corners. In other embodiments, the outer surface of the endplates may have a convex radius from edge to edge swept from front to back, and a separate, and possibly different radius, convex surface swept edge to edge from right to left (discussed in more detail below).

Superior endplate 910 comprises first and second insertion apertures or holes 970 and inferior endplate comprises first and second insertion apertures or holes 972, which are designed to receiving mounting pins or rods of an insertion instrument (not shown). A more complete description of a suitable insertion instrument that cooperates with holes 970, 972 can be found in U.S. Pat. No. 7,547,309, the complete disclosure of which is incorporated herein by reference for all purposes.

Implant 100 further includes a first keel 920 extending from upper bone contacting surface 912 of superior endplate 910 and a second keel 925 extending from the lower bone contacting surface of inferior endplate 940. Keels 920, 925 are centrally located on endplates 910, 940 such that they generally extend along a longitudinal axis of the endplates. The longitudinal axis is hereafter defined as the axis generally extending from an anterior portion of the implant to a posterior portion of the prosthesis after insertion of the implant into the intervertebral body. The anterior portion may also be considered the trailing end of the prosthesis and the posterior portion the leading end (with reference to the direction the prosthesis is introduced into the intervertebral space).

Keels 120, 125 each include a leading end 922, a trailing end 924 and side surfaces therebetween. In some embodiments, leading end 922, trailing end 924 and the side surfaces of keels 920, 925 are substantially perpendicular to the bone contacting surfaces of endplates 910, 940, but, in some embodiments, they may also taper inward in the directions away from these bone contacting surfaces such that the outer surface 966 of keels 920, 925 has a smaller width than the base. The outer surface 966 of keels 920, 925 may be substantially parallel to the bone contacting surfaces of endplates 910, 940. In some embodiments, the outer surface 966 of keels 920, 925 comprises one or more steps 968 to provide a jagged surface for gripping into the vertebral bone. Although not shown in FIG. 1E, the leading ends 922 of the keels 920, 925 may be rounded or beveled to facilitate insertion of the keels into cutouts formed in the adjacent vertebrae.

Upper and inferior endplates 910, 940 each include at least one spike such as a plurality of spikes, projecting from the bone facing surfaces 912, 942 of the endplate bodies. In the exemplary embodiment, endplates 910, 940 comprise first and second spikes 980, 982 positioned laterally outward from keels 920, 925. In some embodiments, each spike 980, 982 has a substantially tapered, rectangular, pyramidal or triangular shape and extends up from a base having a rectangular or alternatively shaped footprint at the bone facing surface, to an upper or outer transverse tip. Each surface extends between the base and the tip, and can be connected between the base and the tip as illustrated. The spikes thus define a transverse axis that extends transversely between the outer tip and the bone facing. In some embodiments, the spikes may define recesses therein that extend from a portion of spikes between the base and the tip and into, but not through, endplates 910, 940.

Implant 900 is manufactured and sold by Centinel Spine of West Chester, Pennsylvania under the tradename of Prodisc® L. A more complete description of implant 900 can be found in commonly assigned U.S. Pat. No. 7,547,309, previously incorporated herein by reference.

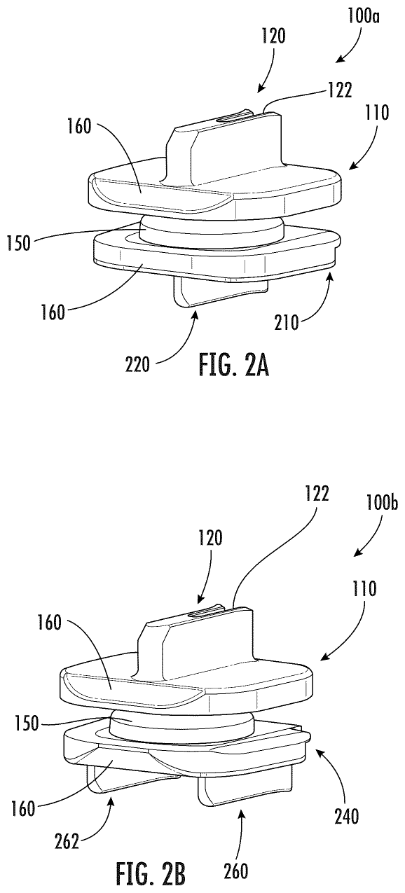

Referring now to FIG. 2A to FIG. 22, spinal implants according to the present disclosure will now be described. As shown in FIG. 2A, a spinal implant 100a comprises an upper or superior endplate 110, a lower or inferior endplate 210 and a core 150. Superior endplate 110 rides upon the core 150 and is operable to rotate relative to core 150, including torsional rotation about a vertical axis, flexion/extension rotation in the posterior/anterior directions and lateral bending rotation. Core 150 includes a substantially spherical dome have a convex upper surface that contacts an internal concave surface of upper endplate 110 and an inlay that is slidably disposed is coupled to lower endplate, e.g., within longitudinal recesses or slots in lower endplate 210. Similar to previous embodiments, the spherical dome may be a separate element from the inlay and coupled thereto, or the core and inlay may be manufactured as a single, integral element.

Upper endplate 110 is substantially similar to upper endplate 110 of implant 100, as shown in FIG. 1A. Lower endplate 220 is substantially similar to lower endplate 210 of implant 200, as shown in FIG. 1B. In some embodiments, upper and lower endplates 110, 210 further include a tapered surface 160 extending from the leading end portion of the endplates that tapers inwardly towards the longitudinal axis to reduce a cross-sectional area of the leading ends and facilitate insertion of the implant into the intervertebral space.

Spinal implant 100A further includes a first keel 120 extending from the upper surface of upper endplate 110 and a second keel 220 extending from the lower surface of lower endplate 210. Keels 120, 220 are centrally located on endplates 110, 210 such that they generally extend along a longitudinal axis of the endplates. Keel 120 is substantially similar to keel 120 of implant 100, as shown in FIG. 1A. Keel 220 is substantially similar to keel 220 of implant 200, as shown in FIG. 1B.

Referring now to FIG. 2B, a spinal implant 100b comprises an upper or superior endplate 110, a lower or inferior endplate 240 and a core 150. Superior endplate 110 rides upon the core 150 and is operable to rotate relative to core 150, including torsional rotation about a vertical axis, flexion/extension rotation in the posterior/anterior directions and lateral bending rotation. Core 150 includes a substantially spherical dome have a convex upper surface that contacts an internal concave surface of upper endplate 110 and an inlay that is slidably disposed within longitudinal recesses in lower endplate 240.

Upper endplate 110 is substantially similar to upper endplate 110 of implant 100, as shown in FIG. 1A. Lower endplate 240 is substantially similar to lower endplate 240 of implant 200, as shown in FIG. 1B. In some embodiments, upper and lower endplates 110, 240 further include a tapered surface 160 extending from the leading end portion of the endplates that tapers inwardly to reduce a cross-sectional area of the leading ends and facilitate insertion of the implant into the intervertebral space.

Implant 100A further includes a first keel 120 extending from the upper surface of upper endplate 110. Keel 120 is centrally located on endplate 110 such that it generally extends along a longitudinal axis of the endplates. Keel 120 is substantially similar to keel 120 of implant 100, shown in FIG. 1A.

Implant 100A further includes second and third keels 260, 262 extending from the lower surface of lower endplate 240. Keels 260, 262 are preferably spaced laterally an equal distance from a longitudinal axis of the implant 100b and are substantially similar to keels 260, 262 of implant 200 shown in FIG. 1B.

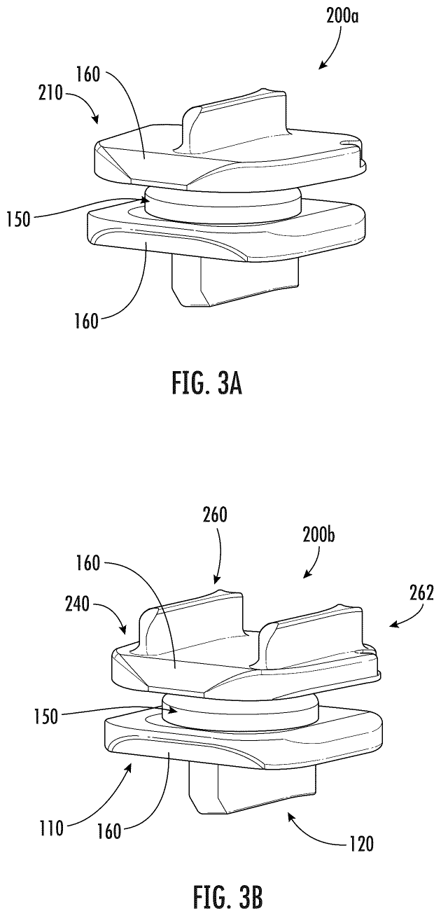

Referring now to FIG. 3A, an intervertebral disc implant 200a comprises an upper or superior endplate 210, a lower or inferior endplate 110 and a core 150. Superior endplate 210 rides upon the core 150 and is operable to rotate relative to core 150, including torsional rotation about a vertical axis, flexion/extension rotation in the posterior/anterior directions and lateral bending rotation. Core 150 includes a substantially spherical dome having a convex upper surface that contacts an internal concave surface of upper endplate 210 and an inlay that is slidably disposed within longitudinal recesses in lower endplate 110.

Upper endplate 210 is substantially similar to upper endplate 210 of implant 200 shown in FIG. 1B. Lower endplate 110 is substantially similar to upper endplate 110 of implant 100 shown in FIG. 1A. In some embodiments, upper and lower endplates 110, 210 further include a tapered surface 160 extending from the leading end portion of the endplates that tapers inwardly to reduce a cross-sectional area of the leading ends and facilitate insertion of the implant into the intervertebral space.

Implant 200a further includes a first keel 220 extending from the bone contacting surface of upper endplate 210. First keel 220 is substantially similar to keel 220 of implant 200, shown in FIG. 1B. Implant 200a further includes a second keel 120 extending from the bone contacting surface of lower endplate 110. Keel 120 is substantially similar to keel 120 of implant 100 shown in FIG. 1A.

Referring now to FIG. 3B, a spinal implant 200b comprises an upper or superior endplate 240, a lower or inferior endplate 110 and a core 150. Superior endplate 240 rides upon the core 150 and is operable to rotate relative to core 150, including torsional rotation about a vertical axis, flexion/extension rotation in the posterior/anterior directions and lateral bending rotation. Core 150 includes a substantially spherical dome having a convex upper surface that contacts an internal concave surface of upper endplate 240 and an inlay that is slidably disposed within longitudinal recesses in lower endplate 110.

Upper endplate 240 is substantially similar to lower endplate 240 of implant 200 shown in FIG. 1B. Lower endplate 110 is substantially similar to upper endplate 110 of implant 100 shown in FIG. 1A. In some embodiments, upper and lower endplates 240, 110 further include a tapered surface 160 extending from the leading end portion of the endplates that tapers inwardly to reduce a cross-sectional area of the leading ends and facilitate insertion of the implant into the intervertebral space.

Implant 200b further includes a first and second keels 260, 262 extending upwardly from the bone contacting surface of upper endplate 240. Keels 260, 262 are substantially similar to keels 260, 262 of implant 200, shown in FIG. 1B, and are preferably spaced laterally an equal distance from a longitudinal axis of the implant 200b. Implant 200b includes a third keel 120 extending downwardly from the bone contacting surface of lower endplate 110. Keel 120 is substantially similar to keel 120 of implant 100 shown in FIG. 1A.

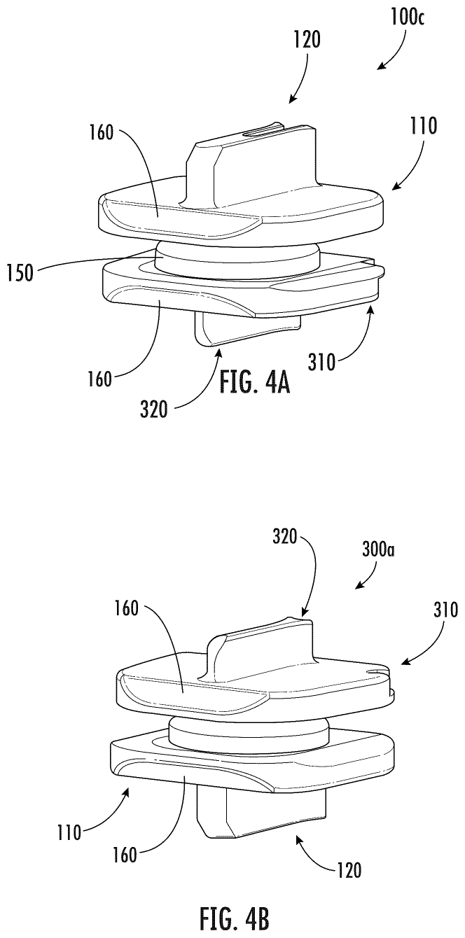

Referring now to FIG. 4A, an implant 100c comprises an upper or superior endplate 110, a lower or inferior endplate 310 and a core 150. Superior endplate 110 rides upon the core 150 and is operable to rotate relative to core 150, including torsional rotation about a vertical axis, flexion/extension rotation in the posterior/anterior directions and lateral bending rotation. Core 150 includes a substantially spherical dome have a convex upper surface that contacts an internal concave surface of upper endplate 110 and an inlay that is slidably disposed within longitudinal recesses in lower endplate 310.

Upper endplate 110 is substantially similar to upper endplate 110 of implant 100 shown in FIG. 1A. Lower endplate 310 is substantially similar to upper endplate 310 of implant 300 shown in FIG. 1C. In some embodiments, upper and lower endplates 110, 310 further include a tapered surface 160 extending from the leading end portion of the endplates that tapers inwardly to reduce a cross-sectional area of the leading ends and facilitate insertion of the implant into the intervertebral space.

Implant 100 further includes a first keel 120 extending from the upper surface of upper endplate 110 and a second keel 320 extending from the lower surface of lower endplate 310. First keel 120 is substantially similar to keel 120 of implant 100 shown in FIG. 1A and second keel 320 is substantially similar to keel 320 of implant 300 shown in FIG. 1C.

Referring now to FIG. 4B, a spinal implant 300a comprises an upper or superior endplate 310, a lower or inferior endplate 110 and a core 150. Superior endplate 310 rides upon the core 150 and is operable to rotate relative to core 150, including torsional rotation about a vertical axis, flexion/extension rotation in the posterior/anterior directions and lateral bending rotation. Core 150 includes a substantially spherical dome have a convex upper surface that contacts an internal concave surface of upper endplate 310 and an inlay that is slidably disposed within longitudinal recesses in lower endplate 110.

Lower endplate 110 is substantially similar to upper endplate 110 of implant 100 shown in FIG. 1A. Upper endplate 310 is substantially similar to upper endplate 310 of implant 300 shown in FIG. 1C. In some embodiments, upper and lower endplates 310, 110 further include a tapered surface 160 extending from the leading end portion of the endplates that tapers inwardly to reduce a cross-sectional area of the leading ends and facilitate insertion of the implant into the intervertebral space.

Implant 100 further includes a first keel 120 extending from the lower surface of lower endplate 110 and a second keel 320 extending from the upper surface of upper endplate 310. First keel 120 is substantially similar to keel 120 of implant 100 shown in FIG. 1A and second keel 320 is substantially similar to keel 320 of implant 300 shown in FIG. 1C.

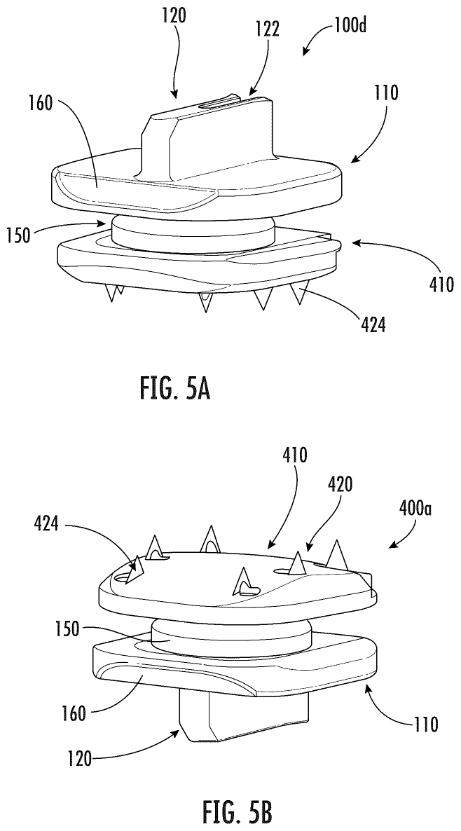

Referring now to FIG. 5A, a spinal implant 100d comprises an upper or superior endplate 110, a lower or inferior endplate 410 and a core 150. Superior endplate 110 rides upon the core 150 and is operable to rotate relative to core 150, including torsional rotation about a vertical axis, flexion/extension rotation in the posterior/anterior directions and lateral bending rotation. Core 150 includes a substantially spherical dome have a convex upper surface that contacts an internal concave surface of upper endplate 110 and an inlay that is slidably disposed within longitudinal recesses or slots in lower endplate 410

Upper endplate 110 is substantially similar to upper endplate 110 of implant 100 shown in FIG. 1A. Lower endplate 410 is substantially similar to upper endplate 410 of implant 400 shown in FIG. 1D. In some embodiments, upper endplate 110 further includes a tapered surface 160 extending from the leading end portion of the endplate that tapers inwardly to reduce a cross-sectional area of the leading end and facilitate insertion of the implant into the intervertebral space.

Implant 100d includes a first keel 120 extending from the upper surface of upper endplate 110. Keel 120 is substantially similar to keel 120 of implant 100. Implant 100d includes a plurality of bone spikes 424 extending downwardly from lower endplate 410. Bone spikes 424 are substantially similar to, and have the same general configuration as, bone spikes 420, 424 of implant 400.

Referring now to FIG. 5B, a spinal implant 400a comprises an upper or superior endplate 410, a lower or inferior endplate 110 and a core 150. Superior endplate 410 rides upon the core 150 and is operable to rotate relative to core 150, including torsional rotation about a vertical axis, flexion/extension rotation in the posterior/anterior directions and lateral bending rotation. Core 150 includes a substantially spherical dome have a convex upper surface that contacts an internal concave surface of upper endplate 410 and an inlay that is slidably disposed within longitudinal recesses or slots in lower endplate 110.

Lower endplate 110 is substantially similar to upper endplate 110 of implant 100 shown in FIG. 1A. Upper endplate 410 is substantially similar to upper endplate 410 of implant 400 shown in FIG. 1D. In some embodiments, lower endplate 110 further includes a tapered surface 160 extending from the leading end portion of the endplate that tapers inwardly to reduce a cross-sectional area of the leading end and facilitate insertion of the implant into the intervertebral space.

Implant 400a includes a first keel 120 extending from the lower surface of lower endplate 110. Keel 120 is substantially similar to keel 120 of implant 100. Implant 400a includes a plurality of bone spikes 420, 424 extending upwardly from upper endplate 410. Bone spikes 420, 424 are substantially similar to, and have the same general configuration as, bone spikes 420, 424 of implant 400.

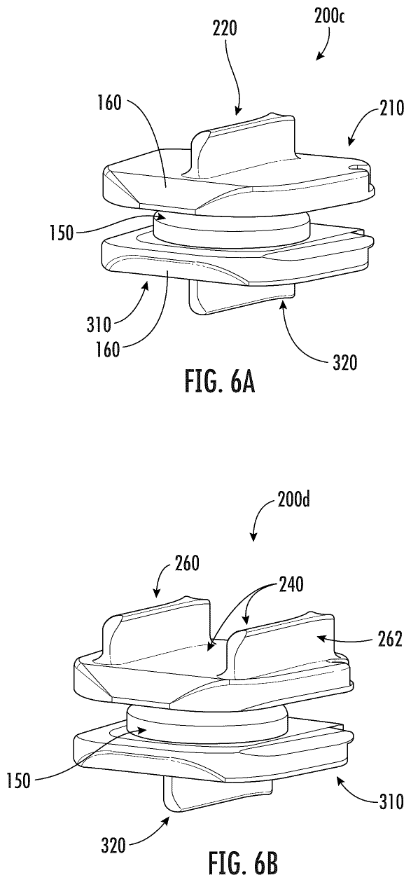

Referring now to FIG. 6A, a spinal implant 200c comprises an upper or superior endplate 210, a lower or inferior endplate 310 and a core 150. Superior endplate 210 rides upon the core 150 and is operable to rotate relative to core 150, including torsional rotation about a vertical axis, flexion/extension rotation in the posterior/anterior directions and lateral bending rotation. Core 150 includes a substantially spherical dome having a convex upper surface that contacts an internal concave surface of upper endplate 210 and an inlay that is slidably disposed within longitudinal recesses in lower endplate 310.

Upper endplate 210 is substantially similar to upper endplate 210 of implant 200 shown in FIG. 1B. Lower endplate 310 is substantially similar to upper endplate 310 of implant 300 shown in FIG. 1C. In some embodiments, upper and lower endplates 210, 310 further include a tapered surface 160 extending from the leading end portion of the endplates that tapers inwardly to reduce a cross-sectional area of the leading ends and facilitate insertion of the implant into the intervertebral space.

Implant 200c further includes a first keel 220 extending from the outer surface of upper endplate 210 and a second keel 320 extending from the lower surface of lower endplate 310. First keel 220 is substantially similar to keel 220 of implant 200. Second keel 320 is substantially similar to keel 320 of implant 300.

Referring now to FIG. 6B, a spinal implant 200d comprises an upper or superior endplate 240, a lower or inferior endplate 310 and a core 150. Superior endplate 240 rides upon the core 150 and is operable to rotate relative to core 150, including torsional rotation about a vertical axis, flexion/extension rotation in the posterior/anterior directions and lateral bending rotation. Core 150 includes a substantially spherical dome having a convex upper surface that contacts an internal concave surface of upper endplate 240 and an inlay that is slidably disposed within longitudinal recesses in lower endplate 310.

Upper endplate 240 is substantially similar to lower endplate 240 of implant 200 shown in FIG. 1B. Lower endplate 310 is substantially similar to upper endplate 310 of implant 300 shown in FIG. 1C. In some embodiments, upper and lower endplates 240, 310 further include a tapered surface 160 extending from the leading end portion of the endplates that tapers inwardly to reduce a cross-sectional area of the leading ends and facilitate insertion of the implant into the intervertebral space.

Implant 200d includes first and second keels 260, 262 extending from the upper surface of upper endplate 240. Keels 260, 262 are substantially similar to keels 260, 262 of implant 200. Implant 200d includes a third keel 320 extending downwardly from the bone contacting surface of lower endplate 310. Keel 320 is substantially similar to keel 320 of implant 300.

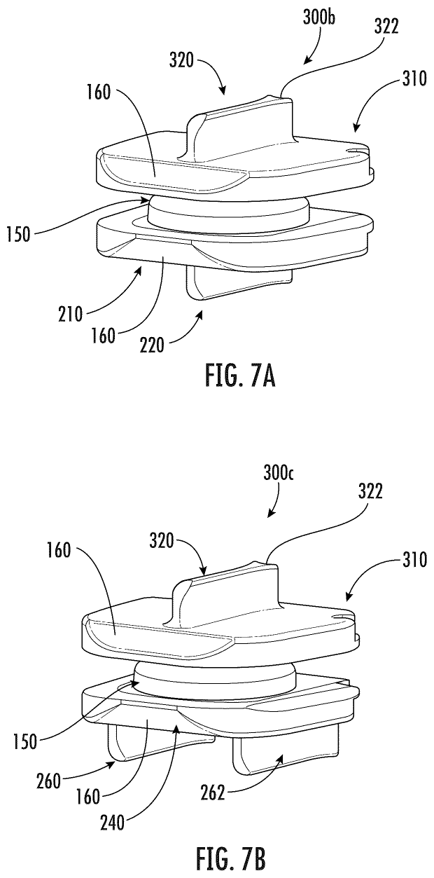

Referring now to FIG. 7A, a spinal implant 300b comprises an upper or superior endplate 310, a lower or inferior endplate 210 and a core 150. Superior endplate 310 rides upon the core 150 and is operable to rotate relative to core 150, including torsional rotation about a vertical axis, flexion/extension rotation in the posterior/anterior directions and lateral bending rotation. Core 150 includes a substantially spherical dome having a convex upper surface that contacts an internal concave surface of upper endplate 310 and an inlay that is slidably disposed within longitudinal recesses in lower endplate 210.

Upper endplate 310 is substantially similar to upper endplate 310 of implant 300 shown in FIG. 1C. Lower endplate 210 is substantially similar to upper endplate 210 of implant 200 shown in FIG. 1B. In some embodiments, upper and lower endplates 310, 210 further include a tapered surface 160 extending from the leading end portion of the endplates that tapers inwardly to reduce a cross-sectional area of the leading ends and facilitate insertion of the implant into the intervertebral space.

Implant 300b includes a first keel 320 extending from the outer surface of upper endplate 310 and a second keel 220 extending from the lower surface of lower endplate 210. First keel 320 is substantially similar to keel 320 of implant 300. Second keel 220 is substantially similar to keel 220 of implant 200.

Referring now to FIG. 7B, a spinal implant 300c comprises an upper or superior endplate 310, a lower or inferior endplate 240 and a core 150. Superior endplate 310 rides upon the core 150 and is operable to rotate relative to core 150, including torsional rotation about a vertical axis, flexion/extension rotation in the posterior/anterior directions and lateral bending rotation. Core 150 includes a substantially spherical dome have a convex upper surface that contacts an internal concave surface of upper endplate 310 and an inlay that is slidably disposed within longitudinal recesses or slots in lower endplate 240.

Upper endplate 310 is substantially similar to upper endplate 310 of implant 300 shown in FIG. 1C. Lower endplate 240 is substantially similar to lower endplate 240 of implant 200 shown in FIG. 1B. In some embodiments, upper and lower endplates 310, 240 further include a tapered surface 160 extending from the leading end portion of the endplates that tapers inwardly to reduce a cross-sectional area of the leading ends and facilitate insertion of the implant into the intervertebral space.

Implant 300c includes first and second keels 260, 262 extending from the lower bone contacting surface of lower endplate 240. Keels 260, 262 are substantially similar to keels 260, 262 of implant 200. Implant 200b includes a third keel 320 extending upwardly from the bone contacting surface of upper endplate 310. Keel 320 is substantially similar to keel 320 of implant 300.

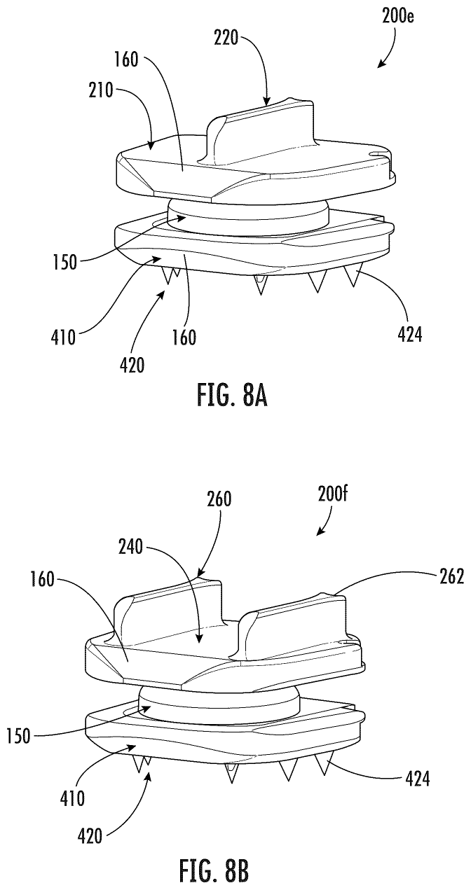

Referring now to FIG. 8A, an intervertebral disc implant 200e comprises an upper or superior endplate 210, a lower or inferior endplate 410 and a core 150. Superior endplate 210 rides upon the core 150 and is operable to rotate relative to core 150, including torsional rotation about a vertical axis, flexion/extension rotation in the posterior/anterior directions and lateral bending rotation. Core 150 includes a substantially spherical dome having a convex upper surface that contacts an internal concave surface of upper endplate 210 and an inlay that is slidably disposed within longitudinal recesses in lower endplate 410.

Upper endplate 210 is substantially similar to upper endplate 210 of implant 200 shown in FIG. 1B. Lower endplate 410 is substantially similar to upper endplate 410 of implant 400 shown in FIG. 1D. In some embodiments, upper endplate 210 further includes a tapered surface 160 extending from the leading end portion of the endplate that tapers inwardly to reduce a cross-sectional area of the leading end and facilitate insertion of the implant into the intervertebral space.

Implant 200e includes a first keel 220 extending from the upper surface of upper endplate 210. Keel 220 is substantially similar to keel 220 of implant 200. Implant 200e includes a plurality of bone spikes 424 extending downwardly from lower endplate 410. Bone spikes 424 are substantially similar to, and have the same general configuration as, bone spikes 420, 424 of implant 400.

Referring now to FIG. 8B, a spinal implant 200f comprises an upper or superior endplate 240, a lower or inferior endplate 410 and a core 150. Superior endplate 240 rides upon the core 150 and is operable to rotate relative to core 150, including torsional rotation about a vertical axis, flexion/extension rotation in the posterior/anterior directions and lateral bending rotation. Core 150 includes a substantially spherical dome having a convex upper surface that contacts an internal concave surface of upper endplate 240 and an inlay that is slidably disposed within longitudinal recesses in lower endplate 410.

Upper endplate 240 is substantially similar to lower endplate 240 of implant 200 shown in FIG. 1B. Lower endplate 410 is substantially similar to upper endplate 410 of implant 400 shown in FIG. 1D. In some embodiments, upper endplate 240 further includes a tapered surface 160 extending from the leading end portion of the endplate that tapers inwardly to reduce a cross-sectional area of the leading end and facilitate insertion of the implant into the intervertebral space.

Implant 200f includes first and second keels 260, 262 extending from the upper surface of upper endplate 240. Keels 260, 262 are substantially similar to keels 260, 262 of implant 200. Implant 200f includes a plurality of bone spikes 424 extending downwardly from lower endplate 410. Bone spikes 424 are substantially similar to, and have the same general configuration as, bone spikes 420, 424 of implant 400.

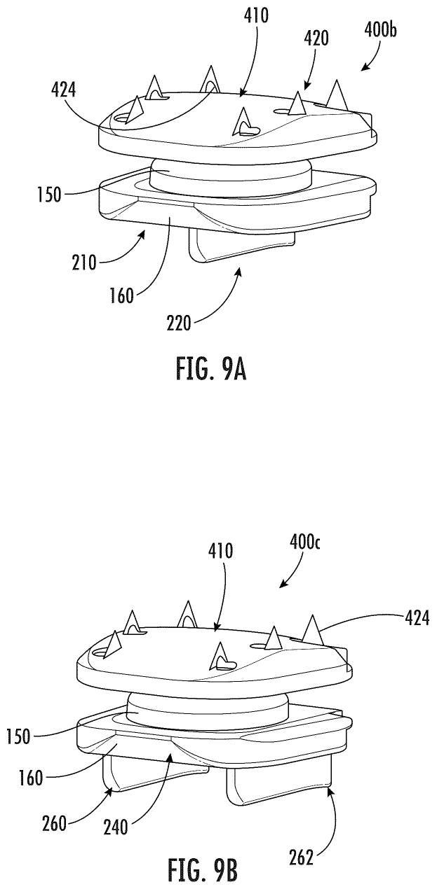

Referring now to FIG. 9A, a spinal implant 400b comprises an upper or superior endplate 410, a lower or inferior endplate 210 and a core 150. Superior endplate 410 rides upon the core 150 and is operable to rotate relative to core 150, including torsional rotation about a vertical axis, flexion/extension rotation in the posterior/anterior directions and lateral bending rotation. Core 150 includes a substantially spherical dome have a convex upper surface that contacts an internal concave surface of upper endplate 410 and an inlay that is slidably disposed within longitudinal recesses in lower endplate 210.

Lower endplate 210 is substantially similar to upper endplate 210 of implant 200 shown in FIG. 1B. Upper endplate 410 is substantially similar to upper endplate 410 of implant 400 shown in FIG. 1D. In some embodiments, lower endplate 210 further includes a tapered surface 160 extending from the leading end portion of the endplate that tapers inwardly to reduce a cross-sectional area of the leading end and facilitate insertion of the implant into the intervertebral space.

Implant 400b includes a keel 220 extending from the lower surface of lower endplate 210. Keel 220 is substantially similar to keel 220 of implant 200. Implant 400b includes a plurality of bone spikes 424 extending upwardly from upper endplate 410. Bone spikes 424 are substantially similar to, and have the same general configuration as, bone spikes 420, 424 of implant 400 shown in FIG. 1D.

Referring now to FIG. 9B, a spinal implant 400c comprises an upper or superior endplate 410, a lower or inferior endplate 240 and a core 150. Superior endplate 410 rides upon the core 150 and is operable to rotate relative to core 150, including torsional rotation about a vertical axis, flexion/extension rotation in the posterior/anterior directions and lateral bending rotation. Core 150 includes a substantially spherical dome having a convex upper surface that contacts an internal concave surface of upper endplate 410 and an inlay that is slidably disposed within longitudinal recesses in lower endplate 240.

Lower endplate 240 is substantially similar to lower endplate 240 of implant 200 shown in FIG. 1B. Upper endplate 410 is substantially similar to upper endplate 410 of implant 400 shown in FIG. 1D. In some embodiments, lower endplate 240 further includes a tapered surface 160 extending from the leading end portion of the endplate that tapers inwardly to reduce a cross-sectional area of the leading end and facilitate insertion of the implant into the intervertebral space.

Implant 400b includes first and second keels 260, 262 extending downwardly from the lower surface of lower endplate 240. Keel keels 260, 262 are substantially similar to keels 260, 262 of implant 200. Implant 400b includes a plurality of bone spikes 424 extending upwardly from upper endplate 410. Bone spikes 424 are substantially similar to, and have the same general configuration as, bone spikes 420, 424 of implant 400 shown in FIG. 1D.

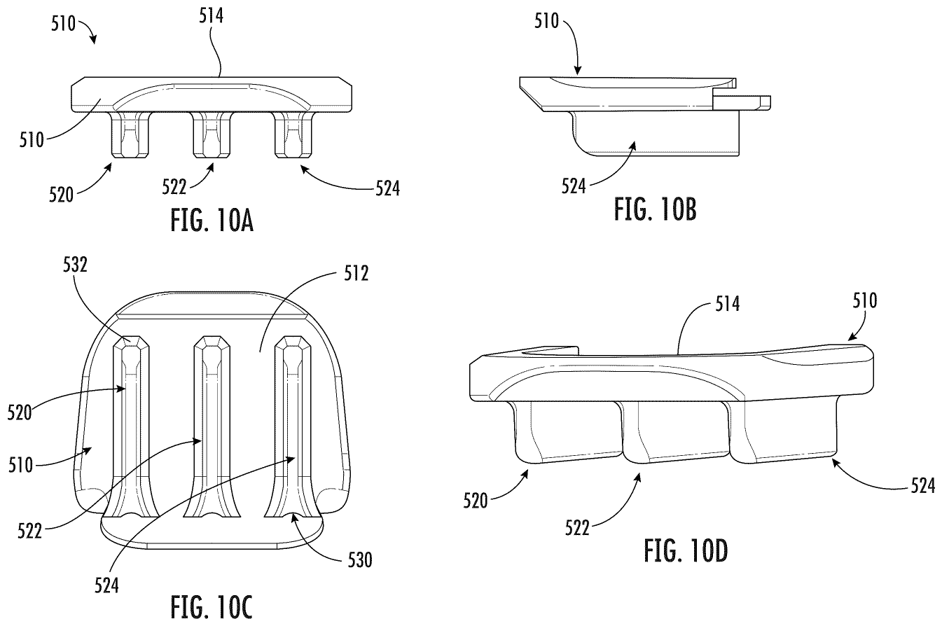

Referring now to FIGS. 10A-10D, another embodiment of an endplate 510 for an intervertebral disc prosthesis will now be described. Endplate 510 may be an upper, superior endplate, a lower inferior endplate or both. As shown, endplate 510 generally has an outer bone contacting surface 512 and an inner surface 514. In some embodiments, inner surface 514 may include an internal concave surface for contacting and articulating with a substantially spherical dome of an articulating core member (not shown). In other embodiments, inner surface 514 may comprises engagement members, such as longitudinal recesses or slots, for coupling with the core, e.g., coupling to an inlay that includes a plastic snap-in projection that fits within snap-in recesses or slots in inner surface 514.

Endplate 510 includes first, second and third keels 520, 522, 524 extending from outer bone contacting surface 512. The keels generally extend in a longitudinal direction, i.e., from the trailing end to the leading end of endplate 510. Second keel 522 generally extends along the longitudinal axis of endplate 510 and first and third keels 520, 524 are laterally offset from the longitudinal axis. In some embodiments, first and third keels 520, 524 are spaced substantially equal distances from the longitudinal axis. In other embodiments, first and third keels 520, 524 may be spaced a different distance from the longitudinal axis (i.e., non-symmetrical).

Keels 520, 522, 524 may have the same configuration as any of the keels described above and shown in FIGS. 1A-1C. Alternatively, keels 520, 522, 524 may have a different configuration from those keels described above. In some embodiments, keels 520, 522, 524 each include a recess or cutout on a trailing end 530 and a rounded or beveled leading end 532.

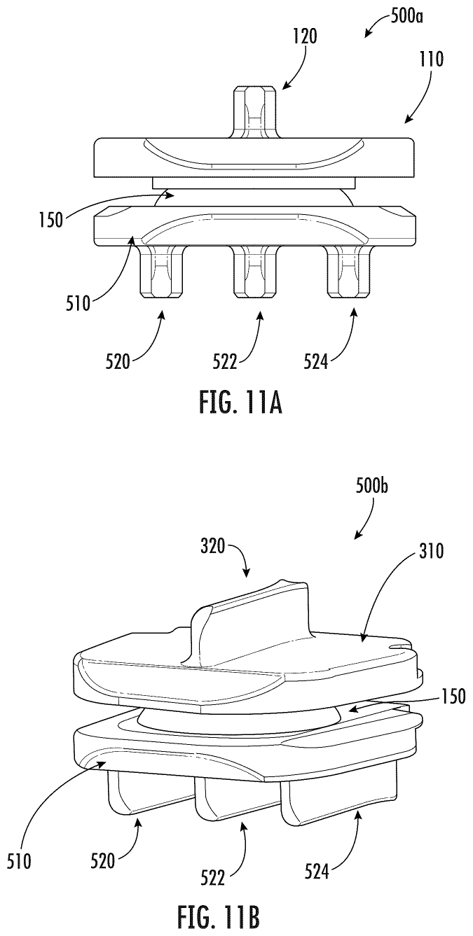

Referring now to FIG. 11A, a spinal implant 500a comprises an upper or superior endplate 110, a lower or inferior endplate 510 and a core 150. Superior endplate 110 rides upon the core 150 and is operable to rotate relative to core 150, including torsional rotation about a vertical axis, flexion/extension rotation in the posterior/anterior directions and lateral bending rotation. Core 150 includes a substantially spherical dome having a convex upper surface that contacts an internal concave surface of upper endplate 110 and an inlay that is slidably disposed within longitudinal recesses in lower endplate 510.

Upper endplate 110 is substantially similar to upper endplate 110 of implant 100 shown in FIG. 1A, although it will be understood that upper endplate 110 may comprise any configuration similar to any of the endplates described above, including endplates 210, 310 and 410 described in FIGS. 1B-1D. Lower endplate 510 is substantially similar to lower endplate 510 shown in FIGS. 10A-10D.

Referring now to FIG. 11B, a spinal implant 500b comprises an upper or superior endplate 310, a lower or inferior endplate 510 and a core 150. Superior endplate 110 rides upon the core 150 and is operable to rotate relative to core 150, including torsional rotation about a vertical axis, flexion/extension rotation in the posterior/anterior directions and lateral bending rotation. Core 150 includes a substantially spherical dome having a convex upper surface that contacts an internal concave surface of upper endplate 310 and an inlay that is slidably disposed within longitudinal recesses in lower endplate 510.

Upper endplate 310 is substantially similar to upper endplate 310 of implant 300 shown in FIG. 1C. Lower endplate 510 is substantially similar to lower endplate 510 shown in FIGS. 10A-10D.

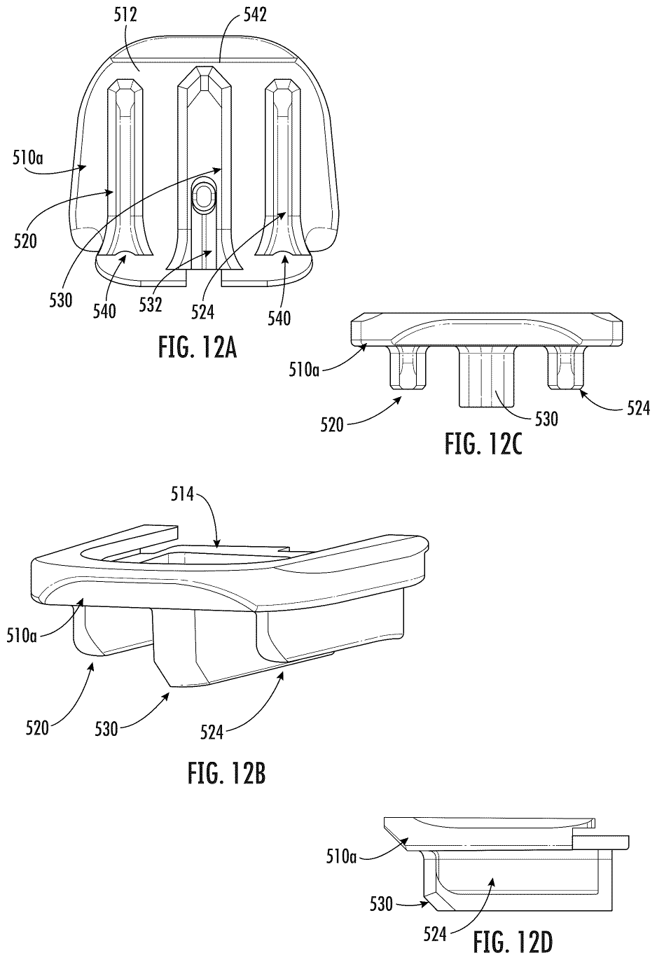

Referring now to FIGS. 12A-12D, another embodiment of an endplate 510a for an intervertebral disc prosthesis will now be described. Endplate 510a may be an upper, superior endplate, a lower inferior endplate or both. As shown, endplate 510a generally has an outer bone contacting surface 512 and an inner surface 514. In some embodiments, inner surface 514 may include an internal concave surface for contacting and articulating with a substantially spherical dome of an articulating core member (not shown). In other embodiments, inner surface 514 may comprises engagement members, such as longitudinal recesses or slots, for coupling with the core, e.g., coupling to an inlay that includes a plastic snap-in projection that fits within the snap-in recesses or slots in inner surface 514.

Endplate 510a includes first, second and third keels 520, 530, 524 extending from outer bone contacting surface 512. The keels generally extend in a longitudinal direction, i.e., from the trailing end to the leading end of endplate 510a. Second keel 530 generally extends along the longitudinal axis of endplate 510a and first and third keels 520, 524 are laterally offset from the longitudinal axis. In some embodiments, first and third keels 520, 524 are spaced substantially equal distances from the longitudinal axis. In other embodiments, first and third keels 520, 524 may be spaced a different distance from the longitudinal axis (i.e., non-symmetrical).

Keels 520, 530, 524 may have the same configuration as any of the keels described above and shown in FIGS. 1A-1C. Alternatively, keels 520, 530, 524 may have a different configuration from those keels described above. In some embodiments, keels 520, 530, 524 each include a recess or cutout on their trailing ends 532, 540 and a rounded or beveled leading end 542. In certain embodiments, leading end 542 of the central keel 530 may have a larger radius of curvature than the leading ends 542 of lateral keels 520, 524. In one such embodiment, leading end 542 of central keel 530 has a substantially arrow shape.

Central keel 530 is generally larger than lateral keels 520, 524. In certain embodiments, central keel 530 extends further away from bone contacting surface 512 of endplate 510 than lateral keels 520, 524 (i.e., a larger depth). Keel 530 may have a depth of about 105% to about 200%, or about 110% to about 150%, or about 120% to about 130%, of the depth of keels 520, 524. In certain embodiments, central keel 530 has a larger thickness or width in the lateral direction than lateral keels 520, 524. Keel 530 may have a width of about 105% to about 200%, or about 110% to about 150%, or about 120% to about 130%, of the width of keels 520, 524. In certain embodiments, central keel 530 is longer in the longitudinal direction than lateral keels 520, 524. Keel 530 may have a length of about 105% to about 200%, or about 110% to about 150%, or about 120% to about 130%, of the length of keels 520, 524.

In various embodiments, central keel 530 is larger in all three dimensions (width, length and depth) than lateral keels 520, 524. In these embodiments, keel 530 may have a volume of about 105% to about 200%, or about 110% to about 150%, or about 120% to about 130%, of the volume of keels 520, 524

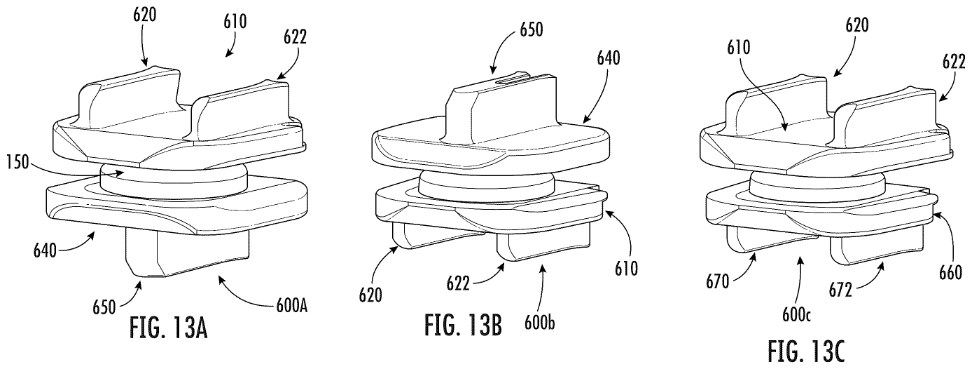

Referring now to FIG. 13A, an intervertebral disc prosthesis 600a comprises an upper or superior endplate 610, a lower or inferior endplate 640 and a core 150. Superior endplate 610 rides upon the core 150 and is operable to rotate relative to core 150, including torsional rotation about a vertical axis, flexion/extension rotation in the posterior/anterior directions and lateral bending rotation. Core 150 includes a substantially spherical dome have a convex upper surface that contacts an internal concave surface of upper endplate 610 and an inlay that is slidably disposed within longitudinal recesses in lower endplate 640.

Upper endplate 610 comprises first and second keels 620, 622 extending upwardly from the bone contacting surface of endplate 610. Keels 620, 622 extend parallel to the longitudinal axis, but are laterally offset from this axis and preferably spaced laterally an equal distance from a longitudinal axis of the implant 600a. Keels 620, 622 may have a configuration similar to any of the above keels (such as keels 120, 220 and 320), or they may have a different configuration from any of the keels described above. In some embodiments, keel 622 has a smaller width, length and/or depth than keel 620. In other embodiments, the width, length and/or depth of keels 620, 622 is substantially the same.

Lower endplate 640 includes a single central keel 650 extending generally along the longitudinal axis of implant 600a. In some embodiments, keel 650 is wider in the lateral direction than keels 620, 622. In some embodiments, keel 650 is longer in the longitudinal direction than keels 620, 622. In some embodiments, keel 650 is deeper than keels 620, 622 (i.e., it extends further away from the endplate). In other embodiments, keel 650 is wider, deeper and longer than keels 620, 622.

Referring now to FIG. 13B, an intervertebral disc prosthesis 600b comprises an upper or superior endplate 640, a lower or inferior endplate 610 and a core 150. Superior endplate 640 rides upon the core 150 and is operable to rotate relative to core 150, including torsional rotation about a vertical axis, flexion/extension rotation in the posterior/anterior directions and lateral bending rotation. Core 150 includes a substantially spherical dome having a convex upper surface that contacts an internal concave surface of upper endplate 640 and an inlay that is slidably disposed within longitudinal recesses in lower endplate 610.

Lower endplate 610 comprises first and second keels 620, 622 extending downwardly from the bone contacting surface of endplate 610. Keels 620, 622 extend parallel to the longitudinal axis, but are laterally offset from this axis and preferably spaced laterally an equal distance from a longitudinal axis of the implant 600b. Keels 620, 622 may have a configuration similar to any of the above keels (such as keels 120, 220 and 320), or they may have a different configuration from any of the keels described above. In some embodiments, keel 622 has a smaller width, length and/or depth than keel 620. In other embodiments, the width, length and/or depth of keels 620, 622 is substantially the same.

Upper endplate 640 includes a single central keel 650 extending generally along the longitudinal axis of implant 600b. In some embodiments, keel 650 is wider in the lateral direction than keels 620, 622. In some embodiments, keel 650 is longer in the longitudinal direction than keels 620, 622. In some embodiments, keel 650 is deeper than keels 620, 622 (i.e., it extends further away from the endplate). In other embodiments, keel 650 is wider, deeper and longer than keels 620, 622.

Referring now to FIG. 13C, an intervertebral disc prosthesis 600c comprises an upper or superior endplate 610, a lower or inferior endplate 660 and a core 150. Superior endplate 610 rides upon the core 150 and is operable to rotate relative to core 150, including torsional rotation about a vertical axis, flexion/extension rotation in the posterior/anterior directions and lateral bending rotation. Core 150 includes a substantially spherical dome having a convex upper surface that contacts an internal concave surface of upper endplate 610 and an inlay that is slidably disposed within longitudinal recesses in lower endplate 660.

Upper endplate 610 comprises first and second keels 620, 622 extending upwardly from the bone contacting surface of endplate 610. Keels 620, 622 extend parallel to the longitudinal axis, but are laterally offset from this axis and preferably spaced laterally an equal distance from a longitudinal axis of the implant 600c. Keels 620, 622 may have a configuration similar to any of the above keels (such as keels 120, 220 and 320). In some embodiments, keel 622 has a smaller width, depth or length than keel 620. In other embodiments, the width, depth or length of keels 620, 622 is substantially the same.

Lower endplate 660 comprises first and second keels 670, 672 extending downwardly from the bone contacting surface of endplate 660. Keels 670, 672 extend parallel to the longitudinal axis, but are laterally offset from this axis and preferably spaced laterally an equal distance from a longitudinal axis of the implant 600c. Keels 670, 672 may have a configuration similar to any of the above keels (such as keels 120, 220 and 320). In some embodiments, keel 672 has a smaller width than keel 670. In other embodiments, the width of keels 670, 672 is substantially the same. In some embodiments, keels 670, 672 have a smaller width than keels 620, 622 of upper endplate 610. In some embodiments, keels 670, 672 are shorter in the longitudinal direction than keels 620, 622. In some embodiments, keels 670, 672 are not as deep as keels 620, 622 (i.e., keels 620, 622 extend further away from the endplate). In other embodiments, keels 620, 622 are wider, deeper and longer than keels 670, 672.

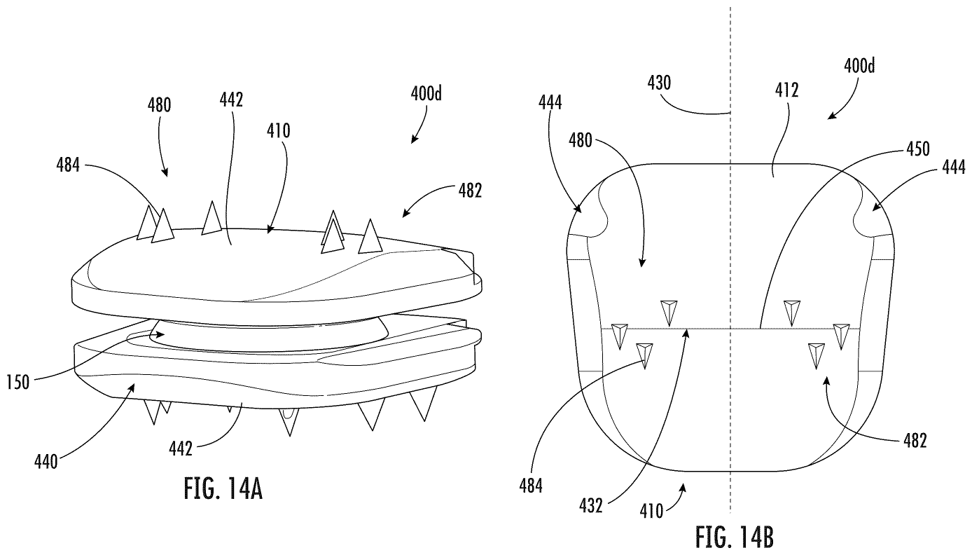

Referring now to FIGS. 14A and 14B, an intervertebral disc prosthesis 400d comprises an upper or superior endplate 410, a lower or inferior endplate 440 and a core 150. Superior endplate 410 rides upon the core 150 and is operable to rotate relative to core 150, including torsional rotation about a vertical axis, flexion/extension rotation in the posterior/anterior directions and lateral bending rotation. Core 150 includes a substantially spherical dome having a convex upper surface that contacts an internal concave surface of upper endplate 410 and an inlay that is slidably disposed within longitudinal recesses in lower endplate 440.

The upper bone contacting surface 412 of upper endplate 410 and the lower bone contacting surface 442 of lower endplate 440 have a substantially domed shape such that the entire contact surfaces are curved. Thus, the contact surfaces are generally convex from an anterior edge of each surface to the posterior edge of each surface. Similarly, the contact surfaces are generally convex from a first lateral edge of the surface to a second lateral edge opposite the first lateral edge. The side surfaces of upper and lower endplates 410, 440 are disposed substantially perpendicularly to the upper surfaces and include rounded or beveled corners. In some embodiments, upper and lower endplates 410, 440 have recesses or notches 444 carved into the two anterior corners.

Upper and lower endplates 410, 440 each include a plurality of spikes 484 projecting from the bone facing surfaces 412, 442 of the endplate bodies. The spikes 484 are arranged in first and second symmetrical and substantially identically constructed groups. One group of spikes 480 is disposed in a first lateral region of the endplate body, and the second group of spikes 482 is disposed in a second lateral region of the endplate body opposite the longitudinal axis 430 from the first group of spikes 480. Each lateral group 480, 482 includes a first or longitudinally forward or front spike, a second or longitudinally middle spike, and a third or longitudinally rear spike, such that the longitudinally middle spike is disposed longitudinally between the forward spike and the rear spike, and forward of the central lateral axis 432. The spikes of each lateral group may be constructed substantially identically and symmetrically. In some embodiments, the spikes in each group are laterally spaced from each other relative to the longitudinal axis. For example, the first or longitudinally forward spike may be disposed laterally between the middle and rear spikes. Alternatively, the first forward spike may be disposed closer or further away from longitudinal axis 430 than the middle and rear spikes.

As shown in FIG. 14B, at least some of the spikes 484 within each lateral group 480, 482 are disposed on opposite sides of an axis 432 extending laterally through a central or mid-line of implant 400d. Central axis 432 is located substantially equal distances from the anterior and posterior ends of prosthesis 400d. In an exemplary embodiment, at least one spike is located on the posterior or leading region of endplates 410, 440 and at least one spike is located in the anterior or trailing region of endplates 410, 440. At least one spike may be located on, or near, central axis 432. In the exemplary embodiment, all of the spikes within each group 480, 482 are located near the central axis 432, i.e., closer to the central axis 432 than the posterior and anterior ends of endplate 410.

Each spike 484 has a substantially pyramidal shape and extends up from a base having a triangular or alternatively shaped footprint at the bone facing surface, to an upper or outer transverse tip. Each surface extends between the base and the tip, and can be connected between the base and the tip as illustrated. The spikes thus define a transverse axis that extends transversely between the outer tip and the bone facing. The spikes define recesses therein that extend from a portion of spikes between the base and the tip and into, but not through, endplates 410, 440.

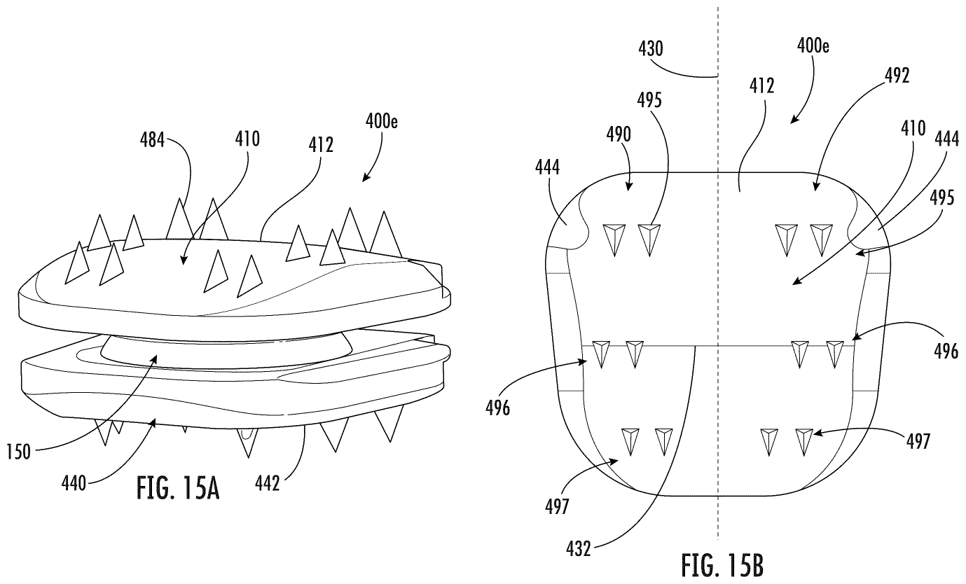

Referring now to FIGS. 15A and 15B, an intervertebral disc prosthesis 400e comprises an upper or superior endplate 410, a lower or inferior endplate 440 and a core 150. Superior endplate 410 rides upon the core 150 and is operable to rotate relative to core 150, including torsional rotation about a vertical axis, flexion/extension rotation in the posterior/anterior directions and lateral bending rotation. Core 150 includes a substantially spherical dome having a convex upper surface that contacts an internal concave surface of upper endplate 410 and an inlay that is slidably disposed within longitudinal recesses in lower endplate 440.

The upper bone contacting surface 412 of upper endplate 410 and the lower bone contacting surface 442 of lower endplate 440 have a substantially domed shape such that the entire contact surfaces are curved. Thus, the contact surfaces are generally convex from an anterior edge of each surface to the posterior edge of each surface. Similarly, the contact surfaces are generally convex from a first lateral edge of the surface to a second lateral edge opposite the first lateral edge. The side surfaces of upper and lower endplates 410, 440 are disposed substantially perpendicularly to the upper surfaces and include rounded or beveled corners. In some embodiments, upper and lower endplates 410, 440 may have recesses or notches 444 carved into the two anterior corners.

Upper and lower endplates 410, 440 each include a plurality of spikes 484 projecting from the bone facing surfaces 412, 442 of the endplate bodies. The spikes 484 are arranged in first and second symmetrical and substantially identically constructed groups. One group of spikes 490 is disposed in a first lateral region of the endplate body, and the second group of spikes 492 is disposed in a second lateral region of the endplate body opposite the longitudinal axis 430 from the first group of spikes 490. Each lateral group 490, 492 includes first and second longitudinally forward or front spikes 497, third and fourth longitudinally middle spikes 496, and fifth and sixth longitudinally rear spikes 495, such that the longitudinally middle spikes 496 are disposed longitudinally between the forward spikes 497 and the rear spikes 495, and forward of the central lateral axis 432. The longitudinally rear spikes 495 are disposed rearward of the central lateral axis 432. The longitudinally middle spikes 496 may be disposed on, or near, central axis 432.

The spikes of each longitudinal group 495, 496, 497 may be constructed substantially identically and symmetrically. In some embodiments, the spikes in each group are laterally spaced from each other relative to the longitudinal axis. In some embodiments, the spikes in each group 495, 496, 497 are longitudinally spaced from each other. In other embodiments, the spikes in each group 495, 496, 497 are aligned with each other in the longitudinal direction (as shown in FIG. 15B).

Each spike 484 has a substantially pyramidal shape and extends up from a base having a triangular or alternatively shaped footprint at the bone facing surface, to an upper or outer transverse tip. Each surface extends between the base and the tip, and can be connected between the base and the tip as illustrated. The spikes thus define a transverse axis that extends transversely between the outer tip and the bone facing. The spikes define recesses therein that extend from a portion of spikes between the base and the tip and into, but not through, endplates 410, 440.

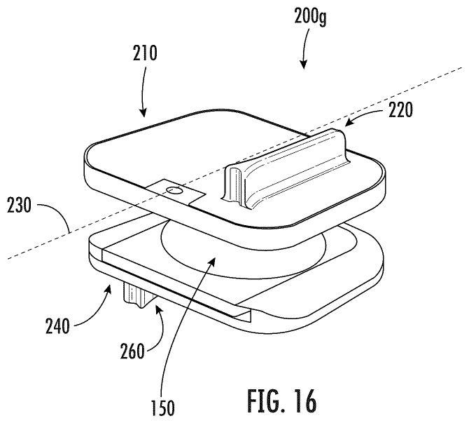

Referring now to FIG. 16, an intervertebral disc prosthesis 200g comprises an upper or superior endplate 210, a lower or inferior endplate 240 and a core 150. Superior endplate 210 rides upon the core 150 and is operable to rotate relative to core 150, including torsional rotation about a vertical axis, flexion/extension rotation in the posterior/anterior directions and lateral bending rotation. Core 150 includes a substantially spherical dome having a convex upper surface that contacts an internal concave surface of upper endplate 210 and an inlay that is slidably disposed within longitudinal recesses in lower endplate 240.

In this embodiment, upper and lower endplates 210, 240 each include a single keel 220, 260 extending away from their bone contacting surfaces. Keels 220, 260 are both laterally spaced away from the longitudinal axis 230 of the implant 200g. In an exemplary embodiment, keel 220 is located on an opposite side of longitudinal axis 230 from keel 260. Keels 220, 260 are preferably spaced equal and opposite distances from longitudinal axis 230.

Keels 220, 260 may have a similar construction as any of the keels described above, or they may have different geometries from the keels described above. In one embodiment, keels 220, 260 are substantially similar to the keels 220, 260, 262 described above in reference to FIG. 1B.

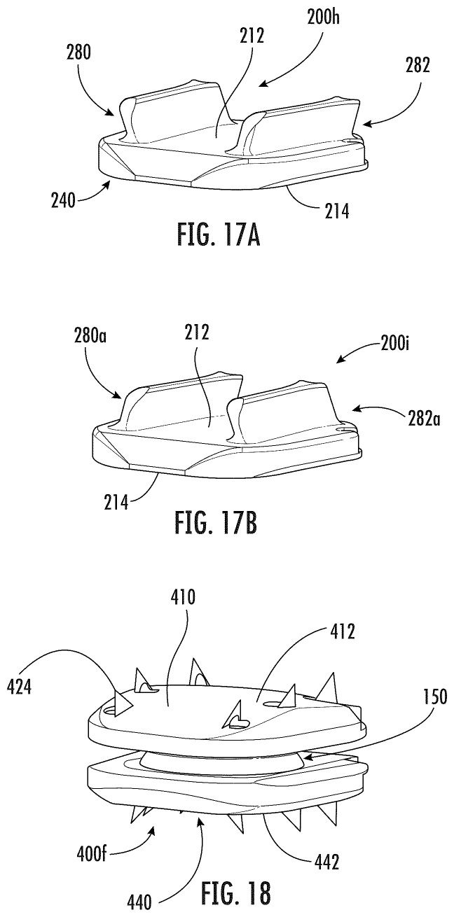

Referring now to FIG. 17A, another embodiment of an endplate 200h for an intervertebral disc prosthesis will now be described. Endplate 200h may be an upper, superior endplate, a lower inferior endplate or both. As shown, endplate 200h generally has an outer bone contacting surface 212 and an inner surface 214. In some embodiments, inner surface 214 may include an internal concave surface for contacting and articulating with a substantially spherical dome of an articulating core member (not shown). In other embodiments, inner surface 214 may comprise longitudinal recesses or slots for coupling with the core, i.e., coupling to an inlay that includes a plastic snap-in projection that fits within the snap-in recesses or slots in inner surface 214.

Endplate 200h includes first and second keels 280, 282 extending away from bone contacting surface 212. As shown, keels 280, 282 are laterally offset from the longitudinal axis of endplate 200h. In addition, keels 280, 282 are oriented at a transverse angle relative to the bone contacting surface 212 such that keels 280, 282 extend in a laterally outward direction (i.e., the outer surface of keels 280, 282 are disposed laterally outward from the base of keels 280, 282).

Referring now to FIG. 17B, another embodiment of an endplate 200i for an intervertebral disc prosthesis will now be described. Endplate 200i may be an upper, superior endplate, a lower inferior endplate or both. As shown, endplate 200i generally has an outer bone contacting surface 212 and an inner surface 214. In some embodiments, inner surface 214 may include an internal concave surface for contacting and articulating with a substantially spherical dome of an articulating core member (not shown). In other embodiments, inner surface 214 may comprise longitudinal recesses or slots for coupling with the core, i.e., coupling to an inlay that includes a plastic snap-in projection that fits within the snap-in recesses or slots in inner surface 214.

Endplate 200 includes first and second keels 280a, 282a extending away from bone contacting surface 212. As shown, keels 280a, 282a are laterally offset from the longitudinal axis of endplate 200i. In addition, keels 280a, 282a are oriented at a transverse angle relative to the bone contacting surface 212 such that keels 280a, 282a extend in a laterally inward direction (i.e., the outer surface of keels 280a, 282a are laterally inward from the base of keels 280a, 282a).

Referring now to FIG. 18, an intervertebral disc implant 400f comprises an upper or superior endplate 410, a lower or inferior endplate 440 and a core 150. Superior endplate 410 rides upon the core 150 and is operable to rotate relative to core 150, including torsional rotation about a vertical axis, flexion/extension rotation in the posterior/anterior directions and lateral bending rotation. Core 150 includes a substantially spherical dome having a convex upper surface that contacts an internal concave surface of upper endplate 410 and an inlay that is slidably disposed within longitudinal recesses or slots in lower endplate 440.

The upper bone contacting surface 412 of upper endplate 410f and the lower bone contacting surface 442 of lower endplate 440f have a substantially domed shape such that the entire contact surfaces are curved. Thus, the contact surfaces are generally convex from an anterior edge of each surface to the posterior edge of each surface. Similarly, the contact surfaces are generally convex from a first lateral edge of the surface to a second lateral edge opposite the first lateral edge. The side surfaces of upper and lower endplates 410, 440 are disposed substantially perpendicularly to the upper surfaces and include rounded or beveled corners. In some embodiments, upper and lower endplates 410, 440 have recesses or notches carved into the two anterior corners.

Upper and lower endplates 410, 440 each include at least one spike 424, such as a plurality of spikes, projecting from the bone facing surfaces 412, 442 of the endplate bodies. The spikes 424 may be arranged in any configuration, such as the configuration shown in FIGS. 1D, 14A, 14B or 15A and 15B, or they may be arranged in a configuration that is not described above. Each spike 424 has a substantially pyramidal shape and extends up from a base having a triangular or alternatively shaped footprint at the bone facing surface, to an upper or outer transverse tip. Each surface extends between the base and the tip, and can be connected between the base and the tip as illustrated. The spikes thus define a transverse axis that extends transversely between the outer tip and the bone facing. The spikes define recesses therein that extend from a portion of spikes between the base and the tip and into, but not through, endplates 410, 440.

In this embodiment, spikes 424 are angled relative to the bone contacting surfaces 412, 442 of the endplates. Thus, the tip of each spike is disposed laterally and/or longitudinally relative to the central portion of the base (i.e., the pyramidal shape does not extend perpendicularly upward or downward from the bone contacting surfaces, but rather extends at a transverse angle relative to the bone contacting surfaces).

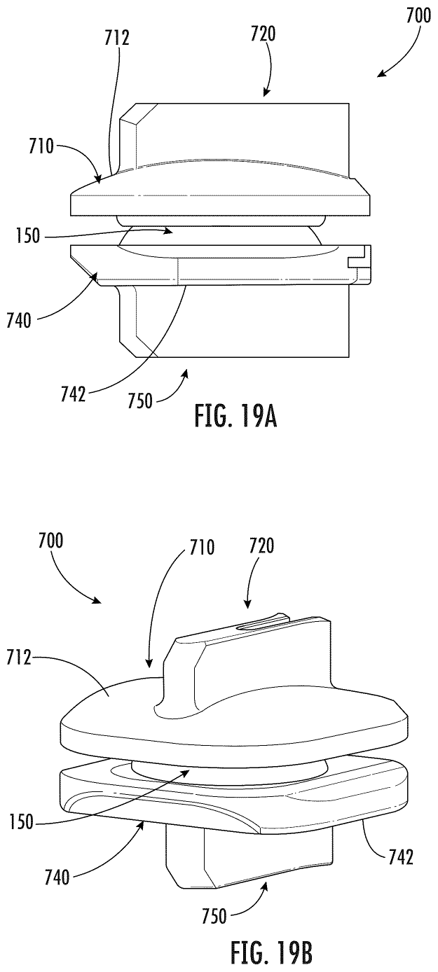

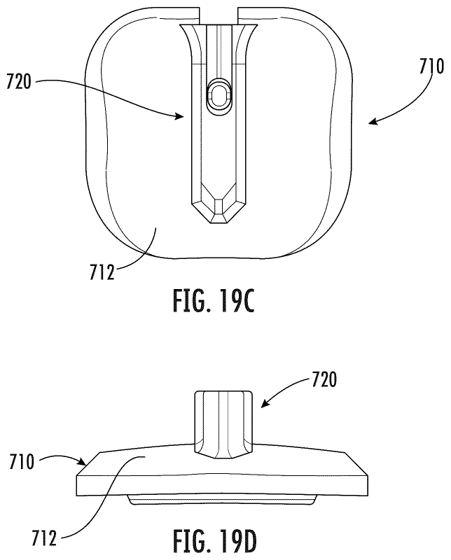

Referring now to FIGS. 19A-19D, an intervertebral disc implant 700 comprises an upper or superior endplate 710, a lower or inferior endplate 740 and a core 150. Superior endplate 710 rides upon the core 150 and is operable to rotate relative to core 150, including torsional rotation about a vertical axis, flexion/extension rotation in the posterior/anterior directions and lateral bending rotation. Core 150 includes a substantially spherical dome having a convex upper surface that contacts an internal concave surface of upper endplate 710 and an inlay that is slidably disposed within longitudinal recesses or slots in lower endplate 740.

Implant 700 further includes a first keel 720 extending from upper bone contacting surface 712 of upper endplate 710 and a second keel 750 extending from lower bone contacting surface 742 of lower endplate 740. Keels 720, 750 are centrally located on endplates 710, 740 such that they generally extend along a longitudinal axis of the endplates. Keels 720, 750 may have a configuration similar to keels 120, 160 shown in FIG. 1A.

The upper bone contacting surface 712 of upper endplate 710 has a substantially domed shape such that the entire contact surface is curved from the apex of the surface to its outer edges. Thus, the contact surface 712 is generally convex from an anterior edge of each surface to the posterior edge of each surface. Similarly, the contact surface 712 may be generally convex from a first lateral edge of the surface to a second lateral edge opposite the first lateral edge.

In some embodiments, the lower bone contacting surface 742 of lower endplate 740 is substantially flat. In other embodiments, lower bone contacting surface 742 may have a substantially domed shaped, similar to upper endplate 710. The side surfaces of upper and lower endplates 710, 740 are disposed substantially perpendicularly to the upper surfaces and include rounded or beveled corners.

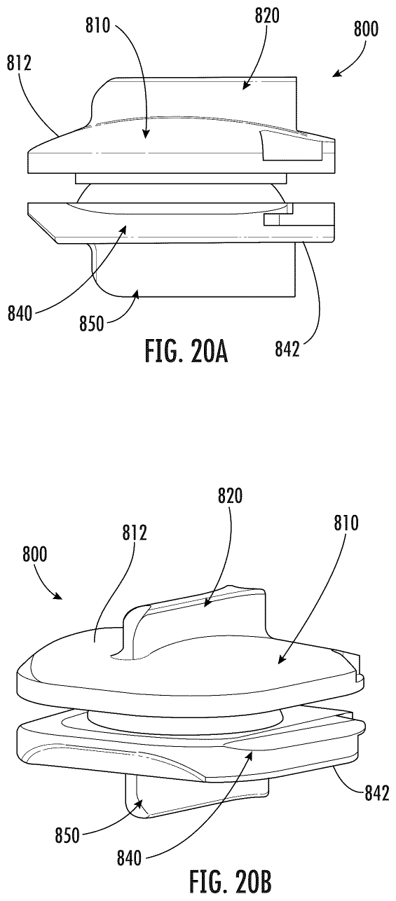

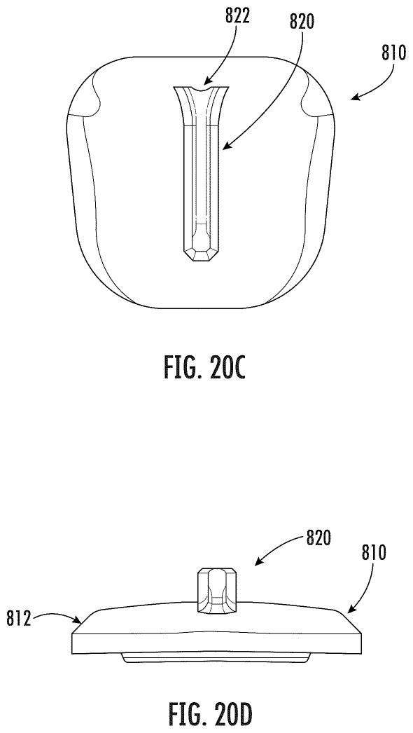

Referring now to FIGS. 20A-20D, an intervertebral disc implant 800 comprises an upper or superior endplate 810, a lower or inferior endplate 840 and a core 150. Superior endplate 810 rides upon the core 150 and is operable to rotate relative to core 150, including torsional rotation about a vertical axis, flexion/extension rotation in the posterior/anterior directions and lateral bending rotation. Core 150 includes a substantially spherical dome having a convex upper surface that contacts an internal concave surface of upper endplate 810 and an inlay that is slidably disposed within longitudinal recesses or slots in lower endplate 840.

Implant 800 further includes a first keel 820 extending from an upper bone contacting surface 812 of upper endplate 810 and a second keel 850 extending from a lower bone contacting surface 842 of lower endplate 840. Keels 820, 850 are centrally located on endplates 810, 840 such that they generally extend along a longitudinal axis of the endplates. Keels 820, 850 may have a configuration similar to keels 220, 260 shown in FIG. 1B.

The upper bone contacting surface 812 of upper endplate 810 has a substantially domed shape such that the entire contact surfaces are curved. Thus, the contact surface 812 is generally convex from an anterior edge of each surface to the posterior edge of each surface. Similarly, the contact surface 812 is generally convex from a first lateral edge of the surface to a second lateral edge opposite the first lateral edge.

In some embodiments, the lower bone contacting surface 842 of lower endplate 840 is substantially flat. In other embodiments, lower bone contacting surface 842 may have a substantially domed shaped, similar to upper endplate 810. The side surfaces of upper and lower endplates 810, 840 are disposed substantially perpendicularly to the upper surfaces and include rounded or beveled corners.

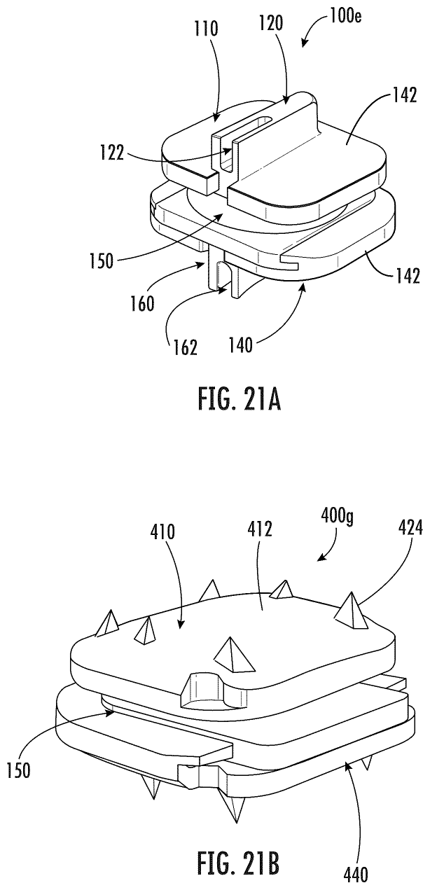

Referring now to FIG. 21A, an intervertebral disc implant 100e comprises an upper or superior endplate 110, a lower or inferior endplate 140 and a core 150. Superior endplate 110 rides upon the core 150 and is operable to rotate relative to core 150, including torsional rotation about a vertical axis, flexion/extension rotation in the posterior/anterior directions and lateral bending rotation. Core 150 includes a substantially spherical dome having a convex upper surface that contacts an internal concave surface of upper endplate 110 and an inlay that is slidably disposed within longitudinal recesses or slots in lower endplate 140. In some embodiments, endplates 110, 140 are substantially the same as endplates 110, 140 in FIG. 1A.

Implant 100s further includes a first keel 120 extending from upper bone contacting surface 112 of upper endplate 110 and a second keel 160 extending from lower bone contacting surface 142 of lower endplate 140. Keels 120, 160 are centrally located on endplates 110, 140 such that they generally extend along a longitudinal axis of the endplates. In some embodiments, keels 120, 160 are substantially the same as keels 120, 160 in FIG. 1A, although it will be recognized that keels 120, 160 may have any suitable shape and geometry, such as that shown in FIG. 1B or 1C.

In this embodiment, endplate 110 has a different cross-sectional area than endplate 140. In the exemplary embodiment, the cross-sectional area of endplate 110 is smaller than the cross-sectional area of endplate 140, preferably about 5% to about 50%, or about 10% to about 30% smaller. Thus, the lateral width of endplate 110 is smaller than the lateral width of endplate 140. In addition, or alternatively, the longitudinal length of endplate 110 is smaller than the longitudinal length of endplate 140.

Referring now to FIG. 21B, an intervertebral disc implant 400g comprises an upper or superior endplate 410, a lower or inferior endplate 440 and a core 150. Superior endplate 410 rides upon the core 150 and is operable to rotate relative to core 150, including torsional rotation about a vertical axis, flexion/extension rotation in the posterior/anterior directions and lateral bending rotation. Core 150 includes a substantially spherical dome having a convex upper surface that contacts an internal concave surface of upper endplate 410 and an inlay that slidably disposed within longitudinal recesses or slots in lower endplate 440. Endplates 410, 440 may be substantially similar to endplates 410, 440 shown in FIG. 1D.

Upper and lower endplates 410, 440 each include a plurality of spikes 424 projecting from the bone facing surfaces 412, 442 of the endplate bodies. The spikes 424 may have a configuration similar to any of the above embodiments.

In this embodiment, endplate 410 has a different cross-sectional area than endplate 440. In the exemplary embodiment, the cross-sectional area of endplate 410 is smaller than the cross-sectional area of endplate 440, preferably about 5% to about 50%, or about 10% to about 30% smaller. Thus, the lateral width of endplate 410 is smaller than the lateral width of endplate 440. In addition, or alternatively, the longitudinal length of endplate 410 is smaller than the longitudinal length of endplate 440.

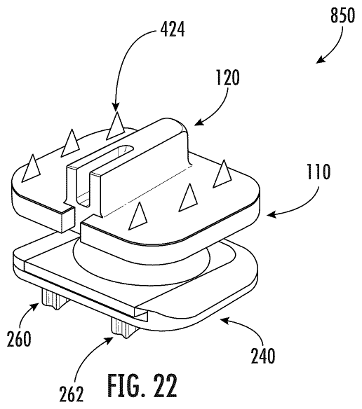

Referring now to FIG. 22, a spinal implant 850 comprises an upper or superior endplate 110, a lower or inferior endplate 240 and a core 150. Superior endplate 110 rides upon the core 150 and is operable to rotate relative to core 150, including torsional rotation about a vertical axis, flexion/extension rotation in the posterior/anterior directions and lateral bending rotation. Core 150 includes a substantially spherical dome having a convex upper surface that contacts an internal concave surface of upper endplate 110 and an inlay that is slidably disposed within longitudinal recesses or slots in lower endplate 240. Endplates 110, 240 may have the same generally configuration as any of the embodiments described above. In an exemplary embodiment, endplate 110 is substantially the same as endplate 110 in FIG. 1A and endplate 240 is substantially the same as endplate 240 in FIG. 1B.

Implant 900 includes a first keel 120 extending from the upper surface of upper endplate 110 and second and third keels 260, 262 extending from the lower bone contacting surface of lower endplate 240. First keel 120 generally extends centrally along the longitudinal axis of implant 900 and second and third keels 260, 262 generally extend parallel to the longitudinal axis, but are laterally offset from this axis. Keels 260, 262 are preferably spaced laterally an equal distance from a longitudinal axis of the implant 900. The keels may have angles, sizes and shapes similar to any of the above embodiments.

Implant 900 further includes a plurality of spikes 424 projecting from the bone facing surface of upper endplate 110. As shown, spikes 424 are disposed laterally outward from keel 120. In an exemplary embodiment, implant 900 includes a group of spikes on each side of keel 120. Each group of spikes may include at least two spikes, such as three spikes, spaced longitudinally from each other. The spikes in each group may also be spaced laterally from each other. The spikes 424 may have angles, sizes and shapes similar to any of the above embodiments.

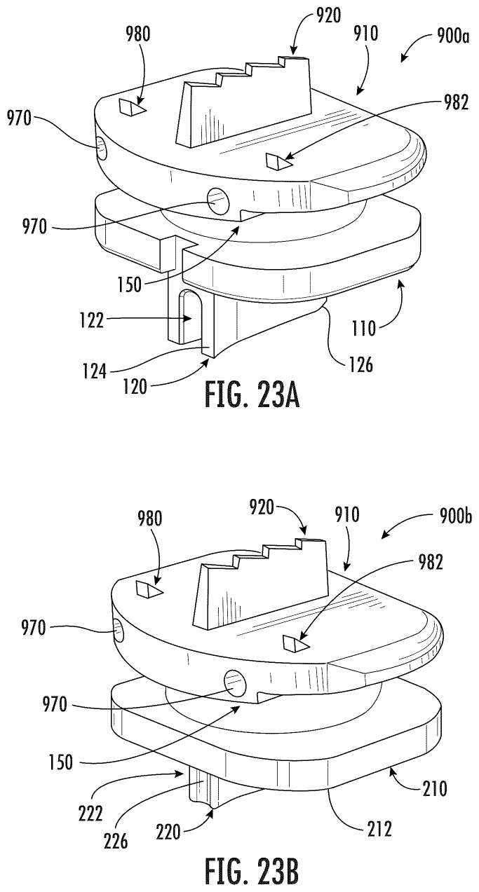

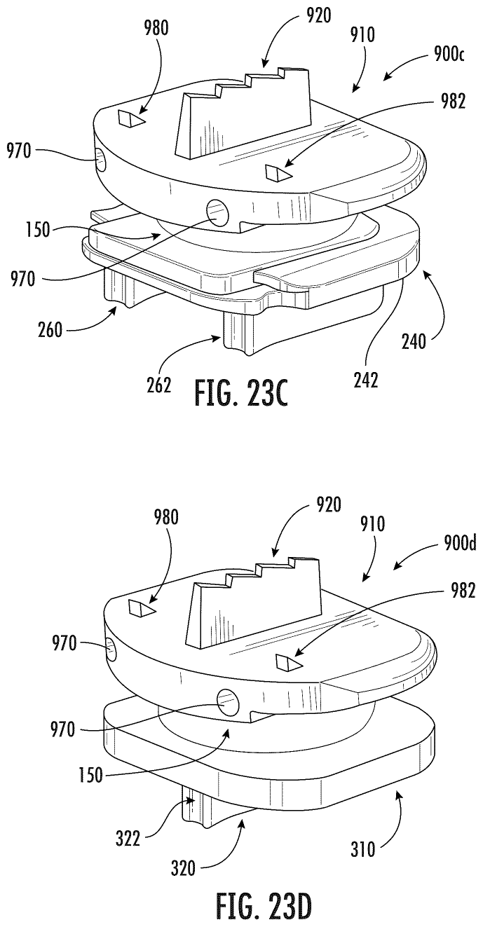

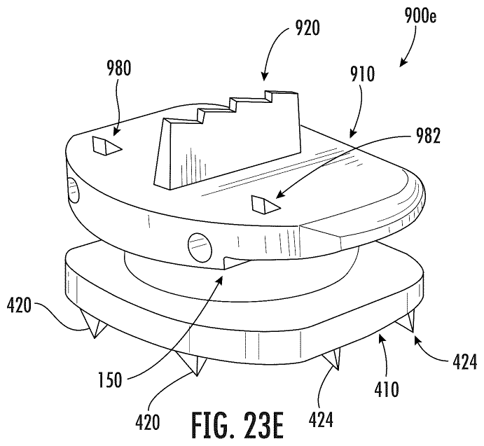

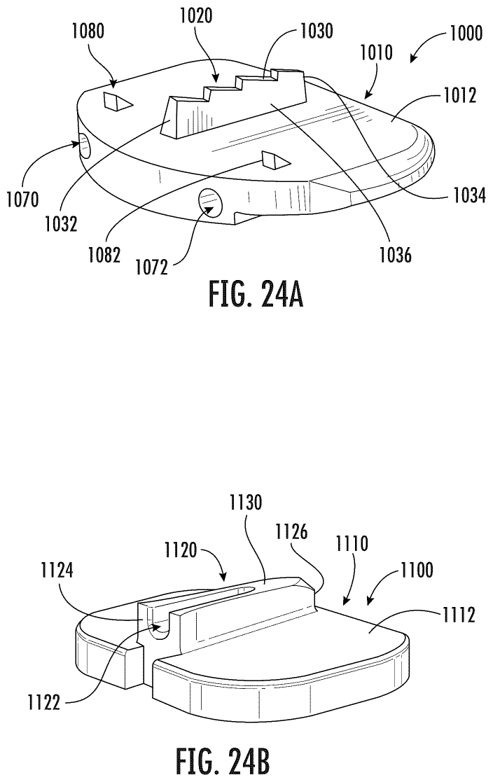

Referring now to FIG. 23A, an intervertebral disc implant 900a comprises an upper or superior endplate 910, a lower or inferior endplate 110 and a core 150. Superior endplate 910 rides upon the core 150 and is operable to rotate relative to core 150, including torsional rotation about a vertical axis, flexion/extension rotation in the posterior/anterior directions and lateral bending rotation. Superior endplate 910 comprises first and second insertion apertures or holes 970, which are designed to receiving mounting pins or rods of an insertion instrument (not shown). Upper endplate 910 further includes a first keel 920 extending from upper bone contacting surface 12 of superior endplate 910. Keel 920 is centrally located on endplates 910 such that it generally extends along a longitudinal axis of the endplates. Endplate 910 comprise first and second spikes 980, 982 positioned laterally outward from keel 920. Superior endplate 920 is substantially similar to the endplate 920 shown in FIG. 1E and described previously.

Inferior or lower endplate 110 comprises a lower bone contacting surface 112 (see FIG. 1A) that is substantially flat and the side surfaces of lower endplate 110 are substantially perpendicular to the lower surface and include rounded or beveled corners. Lower endplate 110 includes a keel 120 extending from lower bone contacting surface 112. Keel 120 is centrally located on endplate 110 such that is generally extends along a longitudinal axis of the endplates. Keel 120 includes a recess 122 that opens upwardly and anteriorly. Recess 122 extends from a trailing end 124 of keel 120 to a portion of keel 120 located between the trailing and leading ends 124, 126 of keel 120 preferably about 25% to about 75% of the distance between the leading and trailing ends, or about 50%. The recess 122 extends from the bone contacting surface 112 of endplate 110 to the upper surface of keel 120 and generally has a width of about 20% to about 50% of the overall width of the keels.

The trailing end 124 and the side surfaces of keel 120 is substantially perpendicular to the bone contacting surface of endplate 110. The outer surface of keel 120 is substantially parallel to the bone contacting surfaces of endplate 110. The leading end 126 of the keel 120 is rounded or beveled to facilitate insertion of the keels into cutouts formed in the adjacent vertebrae. Inferior endplate 120 is substantially similar to the superior endplate 120 shown in FIG. 1A and described previously.

Referring now to FIG. 23B, an intervertebral disc implant 900b comprises an upper or superior endplate 910, a lower or inferior endplate 210 and a core 150. Superior endplate 910 rides upon the core 150 and is operable to rotate relative to core 150, including torsional rotation about a vertical axis, flexion/extension rotation in the posterior/anterior directions and lateral bending rotation. Superior endplate 910 comprises first and second insertion apertures or holes 970, which are designed to receiving mounting pins or rods of an insertion instrument (not shown). Upper endplate 910 further includes a first keel 920 extending from upper bone contacting surface 12 of superior endplate 910. Keel 920 is centrally located on endplates 910 such that it generally extends along a longitudinal axis of the endplates. Endplate 910 comprise first and second spikes 980, 982 positioned laterally outward from keel 920. Superior endplate 920 is substantially similar to the endplate 920 shown in FIG. 1E and described previously.

Inferior endplate 210 comprises a substantially flat lower bone contacting surface 212 having side surfaces that is substantially perpendicular to lower surface 212 and include rounded or beveled corners. Endplate 210 includes a keel 220 extending from lower surface 212. Keel 220 includes an anterior or trailing end 222 that flares laterally outward to anchor this end of the keel in its cutout in adjacent vertebrae. Trailing end 222 may form a concave surface 226 facing in the trailing direction. The posterior or leading end (not shown) of the keel 220 is rounded or beveled to facilitate insertion of the keels into cutouts formed in the adjacent vertebrae. The side surfaces of keel 220 is substantially perpendicular to the bone contacting surface of endplates 210, but, in some embodiments, may also taper inward in the directions away from this bone contacting surface such that the upper surface of keel 220 has a smaller width than the base. The upper surfaces of keel 220 is preferably rounded or beveled to create a smooth curved transition from the side surfaces to the top surfaces. Inferior endplate 210 is substantially similar to the superior endplate 210 shown in FIG. 1B and described previously.

Referring now to FIG. 23C, an intervertebral disc implant 900c comprises an upper or superior endplate 910, a lower or inferior endplate 240 and a core 150. Superior endplate 910 rides upon the core 150 and is operable to rotate relative to core 150, including torsional rotation about a vertical axis, flexion/extension rotation in the posterior/anterior directions and lateral bending rotation. Superior endplate 910 comprises first and second insertion apertures or holes 970, which are designed to receiving mounting pins or rods of an insertion instrument (not shown). Upper endplate 910 further includes a first keel 920 extending from upper bone contacting surface 12 of superior endplate 910. Keel 920 is centrally located on endplates 910 such that it generally extends along a longitudinal axis of the endplates. Endplate 910 comprise first and second spikes 980, 982 positioned laterally outward from keel 920. Superior endplate 920 is substantially similar to the endplate 920 shown in FIG. 1E and described previously.

Lower or inferior endplate 240 comprises a substantially flat lower bone contacting surface 242 having side surfaces that are substantially perpendicular to lower surface 242 and include rounded or beveled corners. Endplate 240 includes first and second keels 260, 262 extending from lower surface 242. First and second keels 260, 262 generally extend parallel to the longitudinal axis, but are laterally offset from this axis. Keels 260, 262 are preferably spaced laterally an equal distance from a longitudinal axis of the implant 900c.

Keel 260, 262 each include an anterior or trailing end that flares laterally outward to anchor this end of the keel in its cutout in adjacent vertebrae. The trailing end may form a concave surface facing in the trailing direction. The posterior or leading end (not shown) of the keel 260, 262 are rounded or beveled to facilitate insertion of the keels into cutouts formed in the adjacent vertebrae. The side surfaces of keels 260, 262 substantially perpendicular to the bone contacting surface of endplate 240, but, in some embodiments, may also taper inward in the directions away from this bone contacting surface such that the upper surfaces of keels 260, 262 have a smaller width than the base. The upper surfaces of keels 260, 262 are preferably rounded or beveled to create a smooth curved transition from the side surfaces to the top surfaces. Inferior endplate 240 is substantially similar to the inferior endplate 240 shown in FIG. 1B and described previously.

Referring now to FIG. 23D, an intervertebral disc implant 900d comprises an upper or superior endplate 910, a lower or inferior endplate 310 and a core 150. Superior endplate 910 rides upon the core 150 and is operable to rotate relative to core 150, including torsional rotation about a vertical axis, flexion/extension rotation in the posterior/anterior directions and lateral bending rotation. Superior endplate 910 comprises first and second insertion apertures or holes 970, which are designed to receiving mounting pins or rods of an insertion instrument (not shown). Upper endplate 910 further includes a first keel 920 extending from upper bone contacting surface 12 of superior endplate 910. Keel 920 is centrally located on endplates 910 such that it generally extends along a longitudinal axis of the endplates. Endplate 910 comprise first and second spikes 980, 982 positioned laterally outward from keel 920. Superior endplate 920 is substantially similar to the endplate 920 shown in FIG. 1E and described previously.

The lower contact surface 312 of lower endplate 310 is substantially flat and the side surfaces of lower endplate 310 are substantially perpendicular to the lower surface and include rounded or beveled corners. Implant 900c further includes a central keel 320 extending from the upper bone contacting surface of lower endplate 310. Keel 320 includes an anterior or trailing end 322 that flares laterally outward to anchor this end of the keel in its cutouts in adjacent vertebrae. Keel 320 extends along the bone contacting surface such that a space exists between the anterior and posterior surfaces of the bone contacting surface of endplates 310 and the keel. The sides surfaces of keel 320 are substantially perpendicular to the bone contacting surface of endplate 310, but, in some embodiments, may also taper inward in the directions away from this bone contacting surface such that the upper portion of keel 320 has a smaller width than the base. The top surface of keel 320 is preferably rounded or beveled to create a smooth curved transition from the side surfaces to the top surface. The posterior or leading end (not shown) of the keel 320 is beveled to facilitate insertion of the keels into cutouts formed in the adjacent vertebrae. Inferior endplate 310 is substantially similar to the superior endplate 310 shown in FIG. 1C and described previously.

Referring now to FIG. 23E, an intervertebral disc implant 900e comprises an upper or superior endplate 910, a lower or inferior endplate 410 and a core 150. Superior endplate 910 rides upon the core 150 and is operable to rotate relative to core 150, including torsional rotation about a vertical axis, flexion/extension rotation in the posterior/anterior directions and lateral bending rotation. Superior endplate 910 comprises first and second insertion apertures or holes 970, which are designed to receiving mounting pins or rods of an insertion instrument (not shown). Upper endplate 910 further includes a first keel 920 extending from upper bone contacting surface 12 of superior endplate 910. Keel 920 is centrally located on endplates 910 such that it generally extends along a longitudinal axis of the endplates. Endplate 910 comprise first and second spikes 980, 982 positioned laterally outward from keel 920. Superior endplate 920 is substantially similar to the endplate 920 shown in FIG. 1E and described previously.

Lower endplate 410 comprises a plurality of spikes, projecting from the bone facing surface. The spikes are arranged in first and second substantially symmetrical and substantially identically constructed groups. One group of spikes 424 is disposed in a first lateral region of the endplate body, and the second group of spikes 420 is disposed in a second lateral region of the endplate body opposite the longitudinal axis from the first group of spikes 424. Each lateral group 420, 424 includes a first or longitudinally forward or front spike, a second or longitudinally middle spike, and a third or longitudinally rear spike, such that the longitudinally middle spike is disposed longitudinally between the forward spike and the rear spike, and forward of the central lateral axis. The spikes of each lateral group can be constructed substantially identically and symmetrically. In some embodiments, the spikes in each group are laterally spaced from each other relative to the longitudinal axis.

Each spike has a substantially pyramidal shape and extends up from a base having a triangular or alternatively shaped footprint at the bone facing surface, to an upper or outer transverse tip. Each surface extends between the base and the tip, and can be connected between the base and the tip as illustrated. The spikes thus define a transverse axis that extends transversely between the outer tip and the bone facing. The spikes define recesses therein that extend from a portion of spikes between the base and the tip and into, but not through, endplate 410. Inferior endplate 410 is substantially similar to the superior endplate 410 shown in FIG. 1D and described previously.

Referring now to FIG. 24A, an intervertebral disc implant 1000 comprises an upper or superior endplate 1010, a lower or inferior endplate (not shown) and a core (not shown). The inferior endplate and the core may be substantially similar to any of the previous embodiments, for example, the embodiment shown in FIG. 1E. Superior endplate 1010 rides upon the core and is operable to rotate relative to the core, including torsional rotation about a vertical axis, flexion/extension rotation in the posterior/anterior directions and lateral bending rotation. Superior endplate 1000 comprises first and second insertion apertures or holes 1070, which are designed to receiving mounting pins or rods of an insertion instrument (not shown). Upper endplate 1010 further includes a first keel 1020 extending from upper bone contacting surface 1012 of superior endplate 1010. Keel 1020 is centrally located on endplate 1010 such that it generally extends along a longitudinal axis of the endplates. Endplate 1010 comprise first and second spikes 1080, 1082 positioned laterally outward from keel 1020. Superior endplate 1020 is substantially similar to the endplate 1020 shown in FIG. 1E and described previously.

In this embodiment, keel 1020 has a smaller height than the keel 920 shown in FIG. 1E. The term “height” is defined herein to mean the distance between upper bone contacting surface 1012 of endplate 1010 and the outer surface 1030 of keel 1020. Applicant has discovered that a shallower keel can provide sufficient purchase and fixation within the vertebral bone without requiring the removal of an excessive amount of bone. In particular, it has been found that a deeper keel does not necessarily improve implant stability or resistance to migration, and in some cases may undesirably weaken the surrounding bone structure or compromise endplate integrity. By contrast, reducing the keel height preserves more of the native bone, maintains greater endplate strength, and can reduce the risk of subsidence or loosening over time. The shorter keel design therefore achieves a favorable balance between fixation strength and bone preservation, resulting in improved long-term stability and reduced risk of mechanical failure.

In embodiments, keel 1020 is less than about 75% or less than about 50% of the height of keel 920. In an exemplary embodiment, the ratio between the height of keel 1020 and the thickness or height of anterior face of the superior endplate 1010 is about 4 mm to about 20 mm, or about 2 mm to about 10 mm, or about 3 mm to about 6 mm, or about 4 mm to about 7 mm.

Referring now to FIG. 24B, an intervertebral disc implant 1100 comprises an upper or superior endplate 1110, a lower or inferior endplate (not shown) and a core (not shown). The inferior endplate and the core may be substantially similar to any of the previous embodiments, for example, the embodiment shown in FIG. 1A. Superior endplate 1110 rides upon the core and is operable to rotate relative to the core, including torsional rotation about a vertical axis, flexion/extension rotation in the posterior/anterior directions and lateral bending rotation.

Superior endplate 1110 includes a keel 1120 extending from an upper bone contacting surface 1112 of endplate 1110. Similar to the embodiment of FIG. 1A, keel 1120 is centrally located on endplate 1110 such that it generally extends along its longitudinal axis. Keel 1120 includes a recess 1122 that opens upwardly and anteriorly and extends from a trailing end 1124 of keel to a portion of keel 112 located between the trailing and leading ends, 1124, 1126.

In this embodiment, keel 1120 has a smaller height than the keel 120 shown in FIG. 1A. The term “height” is defined herein to mean the distance between upper bone contacting surface 1112 of endplate 1110 and the outer surface 1130 of keel 1120. In embodiments, keel 1120 is less than about 75% or less than about 50% of the height of keel 120. In an exemplary embodiment, the ratio between the height of keel 1120 and the thickness or height of anterior portion of the superior endplate 1110 is about 4:1, or about 3:1, or about 3:2 or about 3:1.5

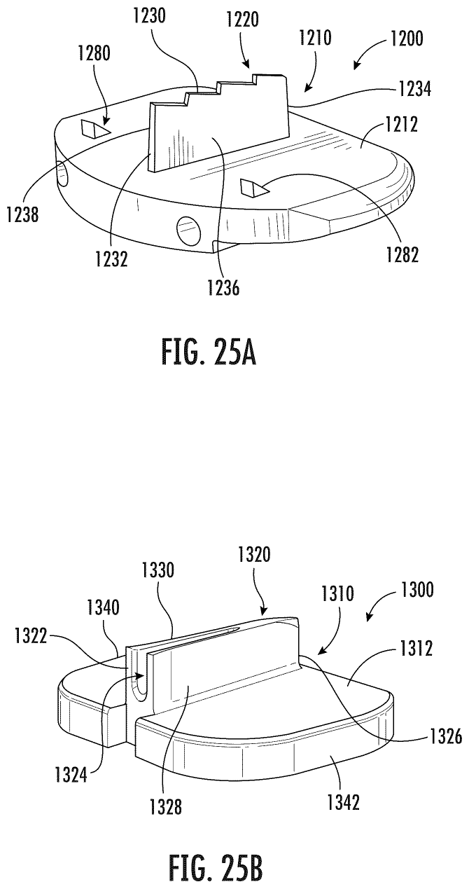

Referring now to FIG. 25A, an intervertebral disc implant 1200 comprises an upper or superior endplate 1210, a lower or inferior endplate (not shown) and a core (not shown). The inferior endplate and the core may be substantially similar to any of the previous embodiments, for example, the embodiment shown in FIG. 1E. Superior endplate 1210 rides upon the core and is operable to rotate relative to the core, including torsional rotation about a vertical axis, flexion/extension rotation in the posterior/anterior directions and lateral bending rotation. Superior endplate 1200 comprises first and second insertion apertures or holes 1270, which are designed to receiving mounting pins or rods of an insertion instrument (not shown). Upper endplate 1210 further includes a first keel 1220 extending from upper bone contacting surface 1212 of superior endplate 1210. Keel 1220 is centrally located on endplate 1210 such that it generally extends along a longitudinal axis of the endplates. Endplate 1210 comprise first and second spikes 1280, 1282 positioned laterally outward from keel 1220. Superior endplate 1220 is substantially similar to the endplate 1220 shown in FIG. 1E and described previously.

In this embodiment, keel 1220 has a smaller width than the keel 920 shown in FIG. 1E. The term “width” is defined herein to mean the lateral distance between side surfaces 1236, 1238, wherein the term “lateral” means substantially perpendicular to the longitudinal or anterior/posterior axis (as defined above). Applicant has discovered that a narrower keel can provide sufficient purchase and fixation within the vertebral bone without requiring the removal of an excessive amount of bone. In particular, it has been found that a wider keel does not necessarily improve implant stability or resistance to migration, and in some cases may undesirably weaken the surrounding bone structure or compromise endplate integrity. By contrast, reducing the keel width preserves more of the native bone, maintains greater endplate strength, and can reduce the risk of subsidence or loosening over time. The narrower keel design therefore achieves a favorable balance between fixation strength and bone preservation, resulting in improved long-term stability and reduced risk of mechanical failure.

In embodiments, keel 1220 is less than about 75% or less than about 50% of the width of keel 920. In an exemplary embodiment, the ratio between the width of keel 1220 and the thickness or height of keel 1220 (measured from upper surface 1212 of endplate 1210 to outer surfaced 1230 of keel 1220) is about 3:8 or about 1:2.

Referring now to FIG. 25B, an intervertebral disc implant 1300 comprises an upper or superior endplate 1310, a lower or inferior endplate (not shown) and a core (not shown). The inferior endplate and the core may be substantially similar to any of the previous embodiments, for example, the embodiment shown in FIG. 1A. Superior endplate 1310 rides upon the core and is operable to rotate relative to the core, including torsional rotation about a vertical axis, flexion/extension rotation in the posterior/anterior directions and lateral bending rotation.

Superior endplate 1310 includes a keel 1320 extending from an upper bone contacting surface 1312 of endplate 1310. Similar to the embodiment of FIG. 1A, keel 1320 is centrally located on endplate 1310 such that it generally extends along its longitudinal axis. Keel 1320 includes a recess 1322 that opens upwardly and anteriorly and extends from a trailing end 1324 of keel to a portion of keel 1320 located between the trailing and leading ends, 1324, 1326.

In this embodiment, keel 1320 has a smaller width than the keel 120 shown in FIG. 1A. The term “width” is defined herein to mean the lateral distance between side surfaces 1328, 1330, wherein the term “lateral” means substantially perpendicular to the longitudinal or anterior/posterior axis (as defined above). In embodiments, keel 1320 is less than about 75% or less than about 50% of the width of keel 120. In an exemplary embodiment, the ratio between the width of keel 1320 and the thickness or height of keel 1320 (measured from upper surface 1312 of endplate 1310 to outer surfaced 1330 of keel 1320) is about 3:8 or about 1:2.

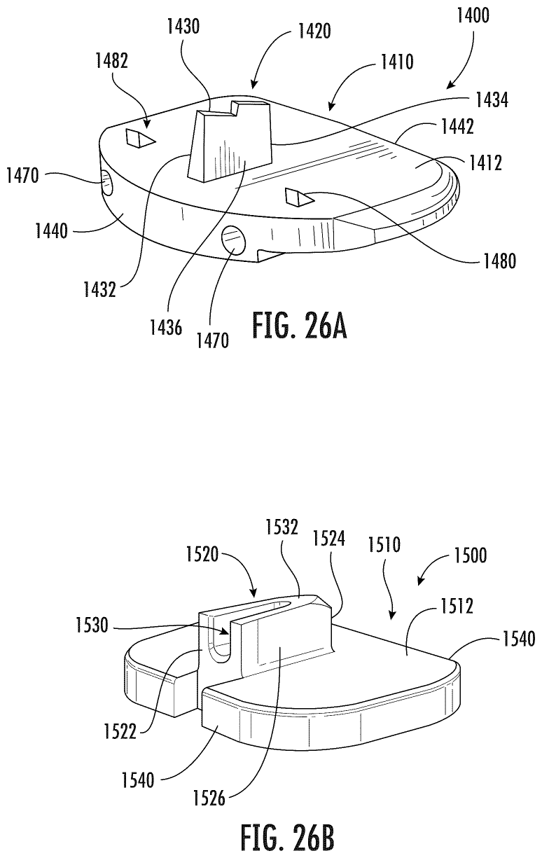

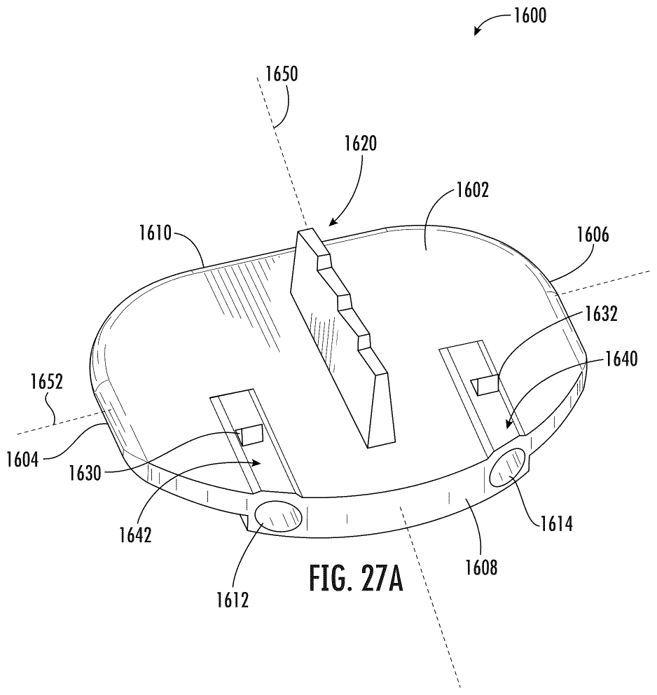

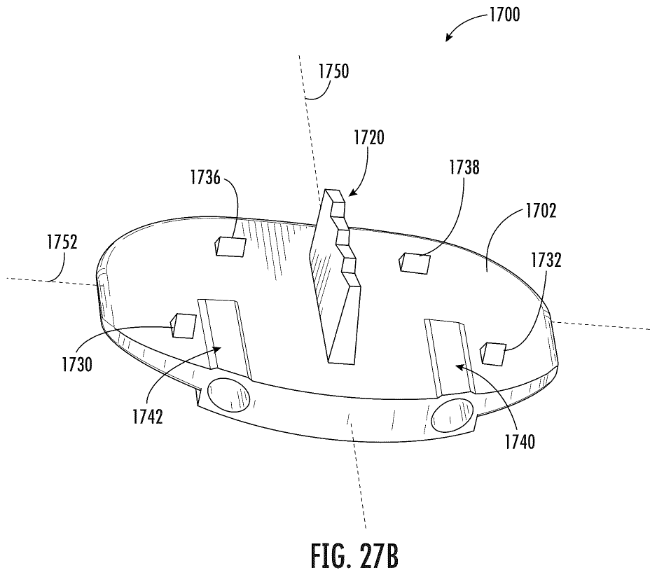

Referring now to FIG. 26A, an intervertebral disc implant 1400 comprises an upper or superior endplate 1410, a lower or inferior endplate (not shown) and a core (not shown). The inferior endplate and the core may be substantially similar to any of the previous embodiments, for example, the embodiment shown in FIG. 1E. Superior endplate 1410 rides upon the core and is operable to rotate relative to the core, including torsional rotation about a vertical axis, flexion/extension rotation in the posterior/anterior directions and lateral bending rotation. Superior endplate 1400 comprises first and second insertion apertures or holes 1470, which are designed to receiving mounting pins or rods of an insertion instrument (not shown). Upper endplate 1410 further includes a first keel 1420 extending from upper bone contacting surface 1412 of superior endplate 1410. Keel 1420 is centrally located on endplate 1410 such that it generally extends along a longitudinal axis of the endplates. Endplate 1410 comprise first and second spikes 1480, 1482 positioned laterally outward from keel 1420. Superior endplate 1420 is substantially similar to the endplate 1420 shown in FIG. 1E and described previously.

In this embodiment, keel 1420 has a smaller length than the keel 920 shown in FIG. 1E. The term “length” is defined herein to mean a distance in the direction of the longitudinal or anterior/posterior axis (as defined above). In embodiments, keel 1420 is less than about 75% or less than about 50% of the length of keel 920.

Referring now to FIG. 26B, an intervertebral disc implant 1500 comprises an upper or superior endplate 1510, a lower or inferior endplate (not shown) and a core (not shown). The inferior endplate and the core may be substantially similar to any of the previous embodiments, for example, the embodiment shown in FIG. 1A. Superior endplate 1510 rides upon the core and is operable to rotate relative to the core, including torsional rotation about a vertical axis, flexion/extension rotation in the posterior/anterior directions and lateral bending rotation.

Superior endplate 1510 includes a keel 1520 extending from an upper bone contacting surface 1512 of endplate 1510. Similar to the embodiment of FIG. 1A, keel 1520 is centrally located on endplate 1510 such that it generally extends along its longitudinal axis. Keel 1520 includes a recess 1522 that opens upwardly and anteriorly and extends from a trailing end 1524 of keel to a portion of keel 1520 located between the trailing and leading ends, 1524, 1526.

In this embodiment, keel 1520 has a smaller length than the keel 120 shown in FIG. 1A. The term “length” is defined herein to mean a distance in the direction of the longitudinal or anterior/posterior axis (as defined above). In embodiments, keel 1520 is less than about 75% or less than about 50% of the length of keel 120.

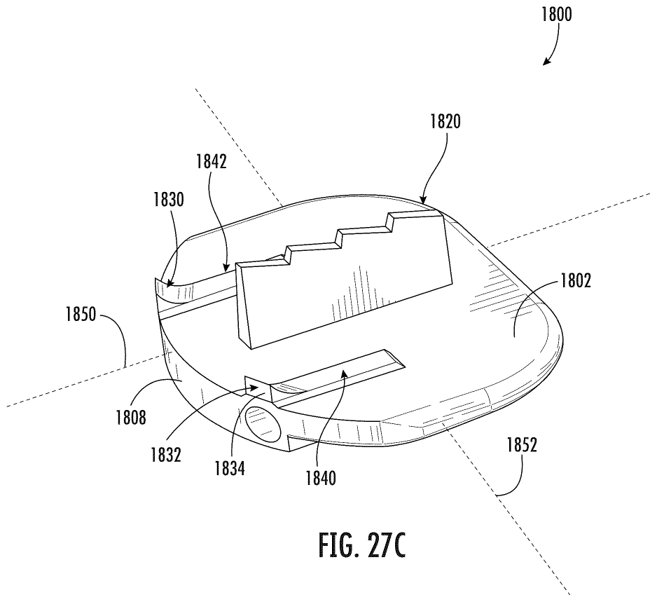

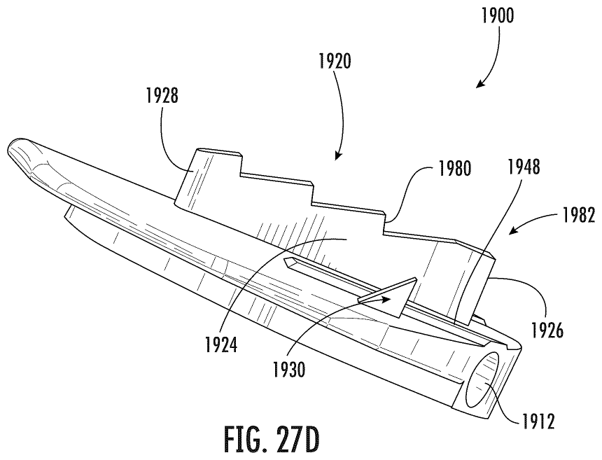

FIGS. 27A-27D illustrate alternative embodiments of an upper endplate that may be used on combination with any of the other upper or lower endplates described herein. The upper endplates in FIGS. 27A-27D are designed such that they have a smaller height or thickness to reduce the overall height of the implant. In certain embodiments, the height of the upper or superior endplate is smaller than the height of inferior endplate (not shown). As used herein, the term “height” of an endplate refers to the distance measured between its outer surface that is configured to contact a vertebral body and its inner surface configured to interface with a core or the opposing endplate of the implant. The reduced-height configuration of the superior endplate offers several clinical and functional advantages. By recessing the concave surface within the superior endplate, the implant maintains a natural articulation profile and full range of motion while occupying less vertical space within the intervertebral disc space. This allows the implant to be more precisely fitted to patients with narrowed or collapsed disc spaces, reducing the need for excessive vertebral distraction during implantation and minimizing the risk of endplate damage. In addition, the lower overall implant height facilitates use in minimally invasive surgical approaches, where limited access and working space can otherwise restrict implant placement. The combination of reduced height and preserved articulation thus improves implant adaptability, surgical flexibility, and patient outcomes across a broader range of anatomical conditions.

Referring now to FIG. 27A, one embodiment of an upper or superior endplate 1600 for use with a spinal implant, such as any of the implants described in the patents referenced above, will now be described. As shown, superior endplate 1600 comprises an upper bone contacting surface 1602 with first and second side walls 1604, 1606, an anterior wall 1608 and a posterior wall 1610.

In this embodiment, upper bone contacting surface 1602 is biconvex, i.e., it is convex in both the direction of the anterior/posterior axis 1650 and the lateral axis 1652. Thus, surface 1602 is convex in the direction of the lateral axis 1652 such that it is has a smaller height (i.e., is thinner) at lateral walls 1604, 1606 than in the center of implant 1600 (i.e., where keel 1620 extends). In addition, surface 1602 is convex in the direction of the anterior/posterior axis 1650 such that it has a smaller height (i.e., is thinner) at posterior and anterior walls 1608, 1610 than in the center of implant 1600 (i.e., where keel 1620 extends). Of course, it will be recognized that upper surface 1602 may have other configurations or shapes. For example, in certain embodiments, upper surface 1602 may be substantially planar (i.e., without a convex shape). In other embodiments, upper surface 1602 may be convex only in the direction of the lateral axis 1652 and relatively planar in the direction of anterior/posterior axis 1650. In yet another embodiment, upper surface 1602 may be convex only in the direction of anterior/posterior axis 1650 and relatively planar in the direction of the lateral axis 1652.

Endplate 1600 includes first and second insertion apertures or holes 1612, 1614 in anterior wall 1608 for receiving mounting pins of an insertion instrument. Upper surface 1602 of superior endplate 1600 further includes first and second raised regions 1640, 1642 extending in the direction of the anterior/posterior axis 1650 and overlying insertion holes 1612, 1614. As discussed below, raised regions 1640, 1642 provide additional material for insertion holes 1612, 1614 with the smaller height of endplate 1600. In particular, raised regions 1640, 1642 allow insertion holes 1612, 1614 to be moved closer to upper surface 1602 of endplate 16200 to accommodate a superior endplate 1600 with a smaller height or thickness. This allows insertion holes 1612, 1614 to pass above the concave surface (not shown of endplate 1600 (i.e., raising holes 1612, 1614 closer to upper surface 1602 provides additional room for these holes between upper surface 1602 and concave surface 1672, which extends into endplate 1602 and therefore reduces the amount of space for the insertion holes).

Raised regions 1640, 1642 preferably extend along the anterior/posterior axis 1650 for all, or substantially all, of the length of insertion holes 1612, 1614. As shown, raised regions 1640, 1642 each comprise a substantially planar outer surface extending to about the midpoint of upper surface 1602 between anterior end 1608 and posterior end 1610, although it will be recognized that raised regions 1640, 1642 can be shorter or longer depending on the length of the mounting pins of the insertion instrument.

In other embodiments, the raised regions may comprise a rounded or convex outer surfaces. For example, the outer surfaces of raised regions may comprise a continuously convex surface from a first lateral side in contact with outer surface 1602 to a second lateral side in contact with outer surface 1602. Alternatively, these outer surfaces may include a substantially planar portion extending between two lateral convex surfaces.

Endplate 1600 includes a central keel 1620 that generally extends along a longitudinal axis of endplate 1600 (i.e., in the direction from the anterior wall 1608 to the posterior wall 1610), and first and second spikes 1630, 1632 spaced laterally from keel 1620 and positioned closer to the anterior wall 1608 than the posterior wall 1610. Keel 1620 may have the same shape and configuration as keel 920 of FIG. 1E (i.e., with a stepped or jagged outer surface) or it may have any other suitable configuration, such as one of the keels described above. Spikes 1630, 1632 are positioned on raised regions 1640, 1642.

FIG. 27B illustrates another embodiment of an upper or superior endplate 1700 for use with a spinal implant, such as any of the implants described in the patents referenced above, will now be described. Similar to the previous embodiment shown in FIG. 27A, superior endplate 1700 comprises an upper bone contacting surface 1702 and first and second insertion apertures or holes 1712, 1714 in the anterior walls for receiving mounting pins of an insertion instrument. Upper surface 1702 of superior endplate 1700 further includes first and second raised regions 1740, 1742 extending in the direction of the anterior/posterior axis 1750 and overlying insertion holes 1712, 1714.

Endplate 1700 includes a central keel 1720 that generally extends along a longitudinal axis of endplate 1700 (i.e., in the direction from the anterior wall to the posterior wall), and first and second spikes 1730, 1732 spaced laterally from keel 1720 and positioned closer to the anterior wall than the posterior wall. Keel 1720 may have the same shape and configuration as keel 920 of FIG. 1E (i.e., with a stepped or jagged outer surface) or it may have any other suitable configuration, such as one of the keels described above.

In this embodiment, endplate 1700 comprises spikes 1730, 1732 positioned laterally outward from raised regions 1740, 1742. Endplate 1700 further includes one or more additional spikes 1736, 1738 positioned posterior to spikes 1730, 1732. In the exemplary embodiment, spikes 1736, 1738 are positioned lateral of axis 1750 and posterior of raised regions 1740, 1742. In certain embodiments, spikes 1736, 1738 may be positioned posterior of keel 1720 or they may be positioned lateral of a posterior portion of keel 1720.

FIG. 27C illustrates another embodiment of an upper or superior endplate 1800 for use with a spinal implant, such as any of the implants described in the patents referenced above, will now be described. Similar to the previous embodiment shown in FIG. 27A, superior endplate 1800 comprises an upper bone contacting surface 102 and first and second insertion apertures or holes 1812, 1814 in the anterior walls for receiving mounting pins of an insertion instrument. Upper surface 1802 of superior endplate 1800 further includes first and second raised regions 1840, 1842 extending in the direction of the anterior/posterior axis 1850 and overlying insertion holes 1812, 1814.

Endplate 1800 includes a central keel 1820 that generally extends along a longitudinal axis of endplate 1800 (i.e., in the direction from the anterior wall to the posterior wall), and first and second spikes 1830, 1832 spaced laterally from keel 1820 and positioned closer to the anterior wall than the posterior wall. Keel 1820 may have the same shape and configuration as keel 920 of FIG. 1E (i.e., with a stepped or jagged outer surface) or it may have any other suitable configuration, such as one of the keels described above.

Spikes 1830, 1832 are positioned on raised regions 1840, 1842 (similar to FIG. 27A). In this embodiment, spikes 1830, 1832 are disposed on an anterior end portion of raised regions 1840, 1842. In particular, spikes 1830, 1832 have an anterior side 1834 that is generally flush with anterior wall 1808 of endplate 1800.

FIG. 27D illustrates another embodiment of an upper or superior endplate 1900 for use with a spinal implant, such as any of the implants described in the patents referenced above, will now be described. Similar to the previous embodiment shown in FIG. 27A, superior endplate 1900 comprises an upper bone contacting surface 1902 and first and second insertion apertures or holes 1912, 1914 in the anterior walls for receiving mounting pins of an insertion instrument. Upper surface 1902 of superior endplate 1900 further includes first and second raised regions 1940 extending in the direction of the anterior/posterior axis and overlying insertion holes 1912.

Endplate 1900 includes a central keel 1920 that generally extends along a longitudinal axis of endplate 1900 (i.e., in the direction from the anterior wall to the posterior wall), and first and second spikes 1930, 1932 spaced laterally from keel 1920 and positioned closer to the anterior wall than the posterior wall. In this embodiment, keel 1920 includes an anterior portion 1982 that flares outward in the anterior direction such that the anterior end 1926 is wider than posterior end 1928 (and wider than the portion of keel 1920 between posterior end 1928 and anterior portion 1982). A wider anterior keel provides a larger surface area for engagement with the vertebral bone in the anterior region, which typically bears greater compressive and shear loads during flexion and extension. This geometry increases resistance to anterior-posterior rocking or micromotion after implantation. Thus, anterior portion 1982 includes first and second side walls that taper outwards in the anterior direction.

Other embodiments will be apparent to those skilled in the art from consideration of the specification and practice of the embodiment disclosed herein. It is intended that the specification and examples be considered as exemplary only, with a true scope and spirit of the embodiment being indicated by the following claims.

For example, in a first aspect, a first embodiment is an implant for insertion into an intervertebral disc space between two adjacent vertebrae. The implant comprises a first component having an outer surface for engaging one of the adjacent vertebrae and an inner surface opposite the outer surface, the first component including a fixation element extending from the outer surface, a second component having an outer surface for engaging the other of the adjacent vertebrae and an inner surface opposite the outer surface, the second component including a fixation element extending from the outer surface, wherein the fixation element of the first component has a different shape than the fixation element of the second component.

A second embodiment is the first embodiment, wherein the fixation element of the first and second components comprises one of a keel or a spike.

A third embodiment is any combination of the previous embodiments, wherein the fixation element of the first component comprises a spike and the fixation element of the second component comprises a keel.

A 4^{th} embodiment is any combination of the previous embodiments, wherein the first component is an upper endplate and the second component is a lower endplate.

A 5^{th} embodiment is any combination of the previous embodiments, wherein the second component is an upper endplate and the first component is a lower endplate.

A 6^{th} embodiment is any combination of the previous embodiments, wherein the fixation element of the first component comprises a spike and wherein the fixation element of the second component comprises a first keel and a second keel.

A 7^{th} embodiment is any combination of the previous embodiments, wherein the first and second keels are laterally offset from a longitudinal axis of the implant.

An 8^{th} embodiment is any combination of the previous embodiments, wherein the fixation element of the first component comprises a spike and wherein the fixation element of the second component comprises a keel, wherein the keel comprises first and second ends, wherein the first end comprises a recess extending from the first end to a location between the first and second ends.

A 9^{th} embodiment is any combination of the previous embodiments, wherein the fixation element of the first component comprises a spike and wherein the fixation element of the second component comprises a keel, wherein the keel comprises a main body with first and second ends that extends along an axis of the endplate, wherein the first end flares laterally outward from the main body of the keel.

A 10^{th} embodiment is any combination of the previous embodiments, wherein the second component is an upper endplate and the first component is a lower endplate.

An 11^{th} embodiment is any combination of the previous embodiments, wherein the first component is an upper endplate and the second component is a lower endplate.

A 12^{th} embodiment is any combination of the previous embodiments, wherein the implant has a longitudinal axis and the fixation element of the first component comprises a first set of spikes laterally offset from the longitudinal axis and a second set of at least two spikes laterally offset from the longitudinal axis on an opposite side of the longitudinal axis.

A 13^{th} embodiment is any combination of the previous embodiments, wherein the first set of spikes comprises at least two spikes offset from each other in a longitudinal direction.

A 14^{th} embodiment is any combination of the previous embodiments, wherein the at least two spikes are laterally offset from each other.

A 15^{th} embodiment is any combination of the previous embodiments, wherein the first set of spikes comprises at least first and second rows of spikes extending in a longitudinal direction, wherein the first and second rows of spikes are laterally offset from each other.

A 16^{th} embodiment is any combination of the previous embodiments, wherein the second set of spikes comprises at least third and fourth rows of spikes extending in a longitudinal direction, wherein the third and fourth rows of spikes are laterally offset from each other.

In another aspect, a first embodiment is an implant for insertion into an intervertebral disc space between two adjacent vertebrae. The implant comprises a first component having an outer surface for engaging one of the adjacent vertebrae and an inner surface opposite the outer surface, the first component including a fixation element extending from the outer surface and a second component having an outer surface for engaging the other of the adjacent vertebrae and an inner surface opposite the outer surface, the second component including a fixation element extending from the outer surface; wherein the fixation element of the first component has a different shape than the fixation element of the second component.

A second embodiment is the first embodiment, wherein the fixation element of the first component comprises a keel and the fixation element of the second component comprises a keel.

A third embodiment is any combination of the previous embodiments, wherein the fixation element of the first component comprises first and second keels laterally offset from a longitudinal axis of the implant.

A 4^{th} embodiment is any combination of the previous embodiments, wherein the fixation element of the second component comprises a keel having a main body with first and second ends that extends along an axis of the endplate, wherein the first end flares laterally outward from the main body.

A 5^{th} embodiment is any combination of the previous embodiments, wherein the fixation element of the second component comprises a keel, wherein the keel comprises first and second ends, wherein the first end comprises a recess extending from the first end to a location between the first and second ends.

A 6^{th} embodiment is any combination of the previous embodiments, wherein the fixation element of the first component comprises a keel, wherein the keel comprises first and second ends, wherein the first end comprises a recess extending from the first end to a location between the first and second ends.

A 7^{th} embodiment is any combination of the previous embodiments, wherein the fixation element of the second component comprises a keel having a main body with first and second ends that extends along an axis of the endplate, wherein the first end flares laterally outward from the main body.

An 8^{th} embodiment is any combination of the previous embodiments, wherein the second component is an upper endplate, and the first component is a lower endplate.

A 9^{th} embodiment is any combination of the previous embodiments, wherein the first component is an upper endplate, and the second component is a lower endplate.

A 10^{th} embodiment is any combination of the previous embodiments, wherein the outer surface of the first component has a substantially domed shape.

An 11^{th} embodiment is any combination of the previous embodiments, wherein the outer surface of the second component has a substantially domed shape.

A 12^{th} embodiment is any combination of the previous embodiments, wherein the outer surface of the second component is substantially flat.

In still another aspect, a first embodiment is an implant for insertion into an intervertebral disc space between two adjacent vertebrae comprising a first component having an outer surface for engaging one of the adjacent vertebrae and an inner surface opposite the outer surface, the first component including a fixation element extending from the outer surface; and a second component having an outer surface for engaging the other of the adjacent vertebrae and an inner surface opposite the outer surface, the second component including a fixation element extending from the outer surface; wherein the fixation element of the first component has a different shape than the fixation element of the second component.

A second embodiment is the first embodiment, wherein the fixation element of the first component comprises at least one spike and at least one keel.

A 3^{rd} embodiment is any combination of the previous embodiments, wherein the fixation element of the second component comprises at least one spike and at least one keel.

A 4^{th} embodiment is any combination of the previous embodiments, wherein the fixation element of the first and second components each comprise a keel and the keel of the first component is laterally offset from the keel of the second component.

A 5^{th} embodiment is any combination of the previous embodiments, wherein the implant includes a longitudinal axis, wherein the fixation element of the first component is laterally offset from the longitudinal axis and the fixation element of the second component is laterally offset from the longitudinal axis on an opposite side of the longitudinal axis from the fixation element of the first component.

A 6^{th} embodiment is any combination of the previous embodiments, wherein the fixation element of the first component comprises a keel with a first width and the fixation element of the second component comprises a keel with a second width, wherein the first width is greater than the second width.

A 7^{th} embodiment is any combination of the previous embodiments, wherein the fixation element of the first component comprises first and second keels with first and second widths and the fixation element of the second component comprises a keel with a third width, wherein the third width is greater than the first and second widths.

An 8^{th} embodiment is any combination of the previous embodiments, wherein the first width is greater than the second width.

A 9^{th} embodiment is any combination of the previous embodiments, wherein the first component comprises first, second and third keels, wherein the first keel extends along a longitudinal axis of the first component and the second and third keels are laterally offset from the longitudinal axis.

A 10^{th} embodiment is any combination of the previous embodiments, wherein the first keel extends a first distance away from the first component and the second and third keels extend a second and third distance, respectively, away from the first component, wherein the first distance is greater than the second and third distances.

An 11^{th} embodiment is any combination of the previous embodiments, wherein the first, second and third keels have first, second and third widths, respectively, wherein the first width is greater than the second and third widths.

A 12^{th} embodiment is any combination of the previous embodiments, wherein the first, second and third keels have first, second and third lengths, respectively, wherein the first length is greater than the second and third lengths.

A 13^{th} embodiment is any combination of the previous embodiments, wherein the first component has first and second keels uniformly spaced across the outer surface of the first component.

A 14^{th} embodiment is any combination of the previous embodiments, wherein the first component has first and second keels non-uniformly spaced across the outer surface of the first component.

A 15^{th} embodiment is any combination of the previous embodiments, wherein the second component has first and second keels uniformly spaced across the outer surface of the second component.

A 16^{th} embodiment is any combination of the previous embodiments, wherein the second component has first and second keels non-uniformly spaced across the outer surface of the second component.

A 17^{th} embodiment is any combination of the previous embodiments, wherein the outer surface of the first component has a first area and the outer surface of the second component has a second area, wherein the first area is less than the second area.

An 18^{th} embodiment is any combination of the previous embodiments, wherein the first component is an upper endplate and the second component is a lower endplate.

In yet another aspect, a first embodiment is an implant for insertion into an intervertebral disc space between two adjacent vertebrae, the implant comprising: a first component having an outer surface for engaging one of the adjacent vertebrae and an inner surface opposite the outer surface, the first component including a fixation element extending from the outer surface; a second component having an outer surface for engaging the other of the adjacent vertebrae and an inner surface opposite the outer surface, the second component including a fixation element extending from the outer surface; wherein the fixation element of the first component has a different shape than the fixation element of the second component; and wherein the outer surface of one of the first and second components has a substantially domed shape.

A second embodiment is the first embodiment, wherein the outer surface of the other of the first and second components is substantially flat.

A 3^{rd} embodiment is any combination of the previous embodiments, wherein the outer surface of the other of the first and second components has a substantially domed shape.

A 4^{th} embodiment is any combination of the previous embodiments, wherein the first component is an upper endplate and the second component is a lower endplate, wherein the outer surface of the upper endplate has a substantially domed shape.

A 5^{th} embodiment is any combination of the previous embodiments, wherein the outer surface of the upper endplate extends from an anterior edge to a posterior edge opposite the anterior edge and is substantially convex from the anterior edge to the posterior edge.

A 6^{th} embodiment is any combination of the previous embodiments, wherein the outer surface of the upper endplate extends from a first lateral edge to a second lateral edge opposite the first lateral edge and is substantially convex from the first lateral edge to the second lateral edge.

A 7^{th} embodiment is any combination of the previous embodiments, wherein the outer surface of the upper endplate comprises an apex and a peripheral edge and is substantially curved from the apex to the peripheral edge.

An 8^{th} embodiment is any combination of the previous embodiments, wherein the fixation element of the first component comprises a spike and the fixation element of the second component comprises a keel.

A 9^{th} embodiment is any combination of the previous embodiments, wherein the fixation element of the first component comprises a spike and wherein the fixation element of the second component comprises a first keel and a second keel.

A 10^{th} embodiment is any combination of the previous embodiments, wherein the fixation element of the first component comprises a keel and the fixation element of the second component comprises a first keel and a second keel.

An 11^{th} embodiment is any combination of the previous embodiments, wherein the fixation element of the first component comprises first and second keels laterally offset from a longitudinal axis of the implant.

A 12^{th} embodiment is any combination of the previous embodiments, wherein the fixation element of the second component comprises a keel having a main body with first and second ends that extends along an axis of the endplate, wherein the first end flares laterally outward from the main body.

A 13^{th} embodiment is any combination of the previous embodiments, wherein the fixation element of the second component comprises a keel having first and second ends, wherein the first end comprises a recess extending from the first end to a location between the first and second ends.

A 14^{th} embodiment is any combination of the previous embodiments, wherein the fixation element of the second component comprises a keel having a main body with first and second ends that extends along an axis of the endplate, wherein the first end flares laterally outward from the main body.

In even still another aspect, a first embodiment is an implant for insertion into an intervertebral disc space between two adjacent vertebrae, the implant having a longitudinal axis and comprising: a first endplate having an outer surface for engaging one of the adjacent vertebrae and an inner surface opposite the outer surface, the first endplate including a first fixation element extending from the outer surface; a second endplate having an outer surface for engaging the other of the adjacent vertebrae and an inner surface opposite the outer surface, the second endplate including a second fixation element extending from the outer surface; and wherein the first endplate has a first height in a direction substantially perpendicular to the longitudinal axis and the first fixation element has a second height in a direction substantially perpendicular to the longitudinal axis, wherein a ratio between the first height and the second height is about 4:3 to about 2:1.

A second embodiment is the first embodiment, wherein the second endplate has a third height in a direction substantially perpendicular to the longitudinal axis and the second fixation element has a fourth height in a direction substantially perpendicular to the longitudinal axis, wherein a ratio between the third height and the fourth height is about 2:1 to about 1:1.

A 3^{rd} embodiment is any combination of the previous embodiments, wherein the first fixation element comprises a keel.

A 4^{th} embodiment is any combination of the previous embodiments, wherein the second fixation element comprises a keel.

In yet still another aspect, a first embodiment is an implant for insertion into an intervertebral disc space between two adjacent vertebrae, the implant having a longitudinal axis and comprising: a first endplate having an outer surface for engaging one of the adjacent vertebrae and an inner surface opposite the outer surface, the first endplate including a first fixation element extending from the outer surface; a second endplate having an outer surface for engaging the other of the adjacent vertebrae and an inner surface opposite the outer surface, the second endplate including a second fixation element extending from the outer surface; and wherein the first endplate has a first width in a direction substantially perpendicular to the longitudinal axis and the first fixation element has a second width in a direction substantially perpendicular to the longitudinal axis, wherein a ratio between the first width and the second width is 3:8.

A second embodiment is the first embodiment, wherein the second endplate has a third width in a direction substantially perpendicular to the longitudinal axis and the second fixation element has a fourth width in a direction substantially perpendicular to the longitudinal axis, wherein a ratio between the third width and the fourth width is about 3:8.

A 3^{rd} embodiment is any combination of the previous embodiments, wherein the first fixation element comprises a keel.

A 4^{th} embodiment is any combination of the previous embodiments, wherein the second fixation element comprises a keel.

In even still another aspect, a first embodiment is an implant for insertion into an intervertebral disc space between two adjacent vertebrae, the implant having a longitudinal axis and comprising: a first endplate having an outer surface for engaging one of the adjacent vertebrae and an inner surface opposite the outer surface, the first endplate including a first fixation element extending from the outer surface; a second endplate having an outer surface for engaging the other of the adjacent vertebrae and an inner surface opposite the outer surface, the second endplate including a second fixation element extending from the outer surface; and wherein the first endplate has a first length in a direction substantially parallel to the longitudinal axis and the first fixation element has a second length in a direction substantially parallel to the longitudinal axis, wherein a ratio between the first length and the second length is about 10:9 to about 2:1.

A second embodiment is the first embodiment, wherein the second endplate has a third length in a direction substantially parallel to the longitudinal axis, and the second fixation element has a fourth length in a direction substantially parallel to the longitudinal axis, wherein a ratio between the third length and the fourth length is about 10:9 to about 3:1.

A 3^{rd} embodiment is any combination of the previous embodiments, wherein the first fixation element comprises a keel.

A 4^{th} embodiment is any combination of the previous embodiments, wherein the second fixation element comprises a keel.

1. An implant for insertion into an intervertebral disc space between two adjacent vertebrae, the implant comprising:
a first component having an outer surface for engaging one of the adjacent vertebrae and an inner surface opposite the outer surface, the first component including a fixation element extending from the outer surface;
a second component having an outer surface for engaging the other of the adjacent vertebrae and an inner surface opposite the outer surface, the second component including a fixation element extending from the outer surface; and
wherein the fixation element of the first component has a different shape than the fixation element of the second component.

2. The implant of claim 1, wherein the fixation element of the first component comprises a spike and the fixation element of the second component comprises a keel.

3. The implant of claim 1, wherein the fixation element of the first component comprises a spike and wherein the fixation element of the second component comprises a first keel and a second keel.

4. The implant of claim 3, wherein the first and second keels are laterally offset from a longitudinal axis of the implant.

5. The implant of claim 1, wherein the fixation element of the first component comprises a spike and wherein the fixation element of the second component comprises a keel, wherein the keel comprises first and second ends, wherein the first end comprises a recess extending from the first end to a location between the first and second ends.

6. The implant of claim 1, wherein the fixation element of the first component comprises a spike and wherein the fixation element of the second component comprises a keel, wherein the keel comprises a main body with first and second ends that extends along an axis of the endplate, wherein the first end flares laterally outward from the main body of the keel.

7. The implant of claim 1, wherein the implant has a longitudinal axis and the fixation element of the first component comprises a first set of spikes laterally offset from the longitudinal axis and a second set of at least two spikes laterally offset from the longitudinal axis on an opposite side of the longitudinal axis.

8. The implant of claim 1, wherein the fixation element of the first component comprises a keel and the fixation element of the second component comprises a keel.

9. The implant of claim 1, wherein the fixation element of the first component comprises first and second keels laterally offset from a longitudinal axis of the implant.

10. The implant of claim 8, wherein the fixation element of the second component comprises a keel having a main body with first and second ends that extends along an axis of the endplate, wherein the first end flares laterally outward from the main body.

11. The implant of claim 8, wherein the fixation element of the second component comprises a keel, wherein the keel comprises first and second ends, wherein the first end comprises a recess extending from the first end to a location between the first and second ends.

12. An implant for insertion into an intervertebral disc space between two adjacent vertebrae, the implant comprising:
a first component having an outer surface for engaging one of the adjacent vertebrae and an inner surface opposite the outer surface, the first component including a fixation element extending from the outer surface;
a second component having an outer surface for engaging the other of the adjacent vertebrae and an inner surface opposite the outer surface, the second component including a fixation element extending from the outer surface;
wherein the fixation element of the first component has a different shape than the fixation element of the second component; and
wherein the outer surface of one of the first and second components has a substantially domed shape.

13. The implant of claim 12, wherein the outer surface of the other of the first and second components is substantially flat.

14. The implant of claim 12, wherein the outer surface of the other of the first and second components has a substantially domed shape.

15. The implant of claim 12, wherein the outer surface of the upper endplate extends from an anterior edge to a posterior edge opposite the anterior edge and is substantially convex from the anterior edge to the posterior edge.

16. The implant of claim 12, wherein the outer surface of the upper endplate extends from a first lateral edge to a second lateral edge opposite the first lateral edge and is substantially convex from the first lateral edge to the second lateral edge.

17. The implant of claim 12, wherein the fixation element of the first component comprises a spike and the fixation element of the second component comprises a keel.

18. The implant of claim 12, wherein the fixation element of the first component comprises a spike and wherein the fixation element of the second component comprises a first keel and a second keel laterally offset from a longitudinal axis of the implant.

19. The implant of claim 12, wherein the fixation element of the second component comprises a keel having a main body with first and second ends that extends along an axis of the endplate, wherein the first end flares laterally outward from the main body.

20. The implant of claim 12, wherein the fixation element of the second component comprises a keel having first and second ends, wherein the first end comprises a recess extending from the first end to a location between the first and second ends.
